SEQUENCE LISTING (18 sequences)

SEQ ID NO: 1 - DNA, 4877 bp, Aspergillus niger
cacgacgggcagagatccgcgacgatggtggtcatcctttgacagaacagatgcgactactcaagtatatttattttatcgtggaactaacttacttcccacaagaattcggcaccaggggaattatactttcagagtcgccttagcccgtatggctgccgactgggcagattcgtgcatggctggaatattggatccatggcgcagataacataatattagggagattgggtgccttcgtccatccaattcctgggtgctccggctacccgacggacaaatctcgcaaaagatagtggagactgttgtcccagttggccagggcaattggacaaagctttccccaataggcagacgtcgccggggaacagagtcccagggtatgaataattcaaccctactactggaatcctggtggcgcttagcgtgcagcagtggatgcactcgccacgaggggaacaggggtcggcttcatccttaatcatttctagaatgcgaccaggatcctcacctgccggtactccaattattggaatttctgcagccggcactgaagataatcaaatgaccgttatggtcttcccacttgatgcagatctacccatgccctacgggggatggtagagagatgatcccttgctctaagctcaccatgcccagcgaccggtacgggaccctcacgtctgtttgaattgatggtgggtcattgtcaaactttgagagaaaaaattcctcggccaacttcaagccacattccagagttgagagacgtcgcagccgacacactcaggaacaaacctttggtgttctggtcggagagagttacgtacaccagcaggggggagggaaggcagatttgaattaaaaatagcggcactagagacacgagaaatgtcagcgacaagtctttctatctcagaccatagaggactcgctagcatgcaccgaagcaaaccgaaggagtcagtcggcagcgtgtgatggatttggcaccgtctgttcgtcgaatccccggaacatccgccgcggtactcatttttagacggaccatcggccacctgccaataaaacggttagcacagctccacttcgtccggttccagtgctcgccgaaagcctaaatcctgccctctggaggttgcatggtgccattctgatccaataccacgcctcatagccactctagcgagcggggcccgaaattacctggaggaagtgccactgtgccatcgcccaactggaagtcgttctccgatcgtgtgtgcctggacgataacccggcccctgtgtcggccagataattccctaaatccatccacaccatctgcctcggcgactcggccagttcctggccatcgctctagtatctctgccacacccagcgtccggctaatccgcggtggtagaattatccaccgcttgctccgcgactttacgcacttggggaacatgtccgatgatggctcaatagcttcctcctgatcgtgagatacggctgctgtatcaatttgcatataaagtacttgtccctccgcagggatcgtcactttattcactcaatccagtcacttcctttctttccatcccctccgtcctcccttagctttacaggatgaaggcttcctggtcttttgcagcatctgtcgctgctctcgcctccaaggccgtcgcttctagcgactgccactgcctgcccggcgatagctgctggccctcgacctccagctgggactccctcaacaacactgtgggcggtcgcttggttgcgactgttcccattggtactccttgccatgaccctaactacaatggcgccgagtgcacgaacctgcaggatgactggtactacccgcagactcagtaaggcaccccctcctccacttcccaaatttaaaccatgctgacgtcagtcaatctcagcttggtctcttcttcatctgttatgcaaccgtactttgccaaccagagctgtgatcctttccagcccgagtcccggccctgcctgcttggcaactacgtgagctacgccgtcaacgtctccacgaccgatgatgttgttgctgcggtcaagttcgcccaggagaacaacatccgtctcgtgatcaggaacaccggtcatgagtaagtttcattttttcccccctctgaatcatgtagattaggatgctaatccatctctcagctaccttggccgttccaccggtgctggtgccctggccatctggactcactacctgaacgacgtcgaaatcacagagtggtcggattccacctacgcgggttcggctgtcaagctgggcagtggtgtcacgggctacaatgtgctcgatgctactcacggaaagggaattgttgtcgtcggcggcgaatgccctactgtcggcctcgctggtggatacaccatgggcggcggtcactccgccctgagcactgccttcggtctgggtgctgaccagaccctgtccttcgaggttgtcaccgcttcgggcgaggtcatcacggcctcccggaccaacaacaccgatctgtactgggccctcagtggtggtggtgccggtaacttcggtgttgtcacctctctcaccgtcaaggcccatcccgatgccaccatctccggtgctgcgctcgaattcaccatcgccaacatcacctccgatctcttctacgaggctgtggagcgcttccacaccttgctgcccgccatggttgatgccggcaccaccgtcatctatgagatgaccaaccaggtcttcctcatcaaccccttgaccgcctacaacaagaccaccgctgaggtcaagaccatcttgtctcccttcctctctgctctgaccgacctgggcatcgagtacaccgtcgcttacacccagtactcctcttactacgaccactacgagaagtacatgggacccctcccctacggcaacctcgaggtcggccagtacaactacggcggccgcctcctcccccgtgacacccttacctcgaacgccgccgatctcgtctccgtcctccgcaacatcacctccgacggtctcatcgccgtcggcgtcggcctgaacgtcaccaactccaacgacaccgccaacgctgtcttccagccctggcgtaacgccgccgtgaccatgcagttcggttccacctggaacgagactgccccttggtccgagatggtcgccgaccagctgcgcattgcccacgactacattccccagttcgaggccgtgacccctggctcgggcgcctacgagaacgagggcagcttccgtcagcagaactggcagaaggaattcttcggcgataactacgcccagctctgcgaggtcaaggagaagtatgatcccgaccatgtcttctacgtcaccaagggtgccggtagcgagtactggtctgtggccgagtcgggtcgcatgtgcaagacccagcaggcgtgcgctgctgtttgaaaaaaaaaatctgtaacataccccgtacgtaatgtttatatgtggttgtttcctgtacatacagtttcgagctagcgctgttgtatagacgatagattacggctttcatagactaataatgtcttggtttttcttacgtgattcataatttatcctctcagtaaacaaaaatacccttaatgttattctcactgagtgaagaccataataaggtagtctcttgtcttttaataaggtcattaggtaataataggcggtatttcatgctcaccagtaacgtcactccaggcagagagagcatcggaatatatttccgtcatgcttccaatcaaagtacatgagccaactagtagaattcgagtgaaacaagactagaagaatgaagatagagaagatatgggaagaaagaaagacagatccgaaggtgcaaatactaccattaggataagatatatatatttctgccggagatctcaattagccctcttgagtactagttaagtactactactaccactaccacaactactactccaatctaactacatatcattatatgtgacagcttaccgttagctaatgtttggaatttctcacaaaaagaaataagggtagatgttgtgattgtggggagggagttagatggagaattgatggcttatgtttggaacaggagacccgtctggtatgcaaggttggtgatactgtcattcttgtgcctgacaggttgaggtccgcgaggtgtgaggatgaagagacaagtatgggtggcattgacatgattcatcagatggaatgggatattcagtgaggataaaggaaaaggtgtaactagtacctggttttgagattgtggtaattggtaggagttctgctgtgttctcgaattgacaatgtatcgtgtaaaaactcatgtacgctgatgattaagtttttgcttgatttgatctctatacccaatttatgtagttttacggatgcttctacataactgaggcccaatctagtctatttatctggtttcgtttagcccacggagcagtgaggggaaggagaggctggttgaggggcatcagtgatatgatctcttttcaattgagagctataataaagtgaccttgtgttttctcctacttggccttataatatcttcacagaaccggatatgtatcgtcttccgtttatggttggcatcggagttgatctgtagttcacgacgagcagagtctaagacatacatggttcagacagagtgtgtaaactacacttgtaggtattgttctctcaaatgtacgaacgtctcacccgttagcatccgccagcatactcgacgaggcgtgtctcagacaccatctagtcaagatgtgacttgaggccttacttcgaggatgcgccaacggagcgatccagtgattgactgaagtcactttc

SEQ ID NO: 2 - DNA, 2852 bp, Aspergillus niger
gtcttgaacaggcggagtccgccctgcaatgcaggcaggcgtagttgcaatatccaggacaattctcccctcttatcacgccgatcgggaagaagcgaagcctcctatagcccaggcagaaacaggagttctgcagccattgaaaatttctactcccacaaaggagagccttggcagcgaaagcaaggtttgccaggcttccttcgtgctccgaacatcctcggcatccaattgacatcaacagactgcaggggttctggtttctgaacggcaaccggccgattcttttaataagttgcaacgtgaatagattgaccctgctccaccccagagtctattcgggcccacctgtaagccctagagagagttagtcctccgagcatccagtcatcccgcgatccccgatgctgcctcgacccacatattaccctcacgtactcaattgaacacggagcagattggacgggatggaccgaccagttccccaagcgagcgccgatggcccgcgagactgcacagggaccgcacgcggcgctcgttggtgctgcataacttctgatcggaaaaccgcaacagagcgatcgggtggagctgcacactgggtatcatatgacgcttgtctcactttgcagagtaactgcaaagtaagattagtgcgctccgtaatctcgcatggcgaaggaatgctagaaataaatgaggtgccccaattgccgggtcagggtcagtccatctgacaaggattccccgggccctccgacgtgcagcagaagggcggtcgcgtggccagtcatcttgccagattccttttttcttatttttgctttttgcattgttcttcttttattcccaccgcatctcattgccgatcgggaattctctgcgccgccatgtttcgactcaagatggaggtgctcacggccatcgcggcctgggcattcgccacggtctcgcagcaaatccctcgctcctccagctgggaaagtgacctccagaccctctcgggtaggctttccaatacctcccagatctattatccgggctccagtggattcacaaatgccaccacgcggtggtccgtcctggatgagcctgaggttaatgttgtcgtggtccctggcaccgaaaatgatgtcgccgaaattgtatgtcaaactcacacttgattgaattgtttcttccccttccatcccccctccccttggaccatagatgtaatgtgacttaattggtcgctaattgatcctgcaggtgaaatttgccaatcagaaggatgtccccttcctaacctacaatggtgtgcacggtgccctcatctctctgggagaaatgacccatggtattgctatctacatgggccagttgagtagtgtcgaggtcgcagctgacggcaagactgccacgatcggaggtggaaccatgtccaaggaggtcaccgaccagctttgggccgcaggaaagcagaccgtgactggaacctgtgaatgtgtcagtcttgtcggtcctgcgcttggtggtggccatggttggctccagggccaccacggccttgttcttgatcagtttgtctcgatgaacatcgtgctggcgaacggcactctgacccacatcgacgccaactcggacctctggtgggccgtcaagggtgccggccacaactttggcatcgtcacttccctcaccatgaagatctatgacatcgagtacagcgattgggccatcgagacgctgaccttcagtggcgacaaggttgccgaggtctaccaggctgccaatgactacctggtcaagaacggcacccaggctgccggcgtgatcaactggtcgtactggatgaacaatgcggatgccgaccccaacaacccggtcatcatcttctacatcatccaggagggagtgaagaccgtcgattccgtctacaccgcgcctttccgcaagatcggtcccatttctgtgtcccccaacaacggcacatacaaggatctcgccgcatggactgaggttgctgtcgactccgccccctgccagaagatgggtatggccaacccccgttacccgatctacctcgaaacgtacaacgtcactgcccagcagaaggcatgggacgtctacgcgaatgctacccgtggcttttcggcgttcaacaactccatcttcatgttcgagggatactcagttggcggcgtgcacgatgttgatagccggtcgagcgcttttgccttccgtaatgaaaacgtgctggctgctcccatgatcaactactaccctgatggcgccgaacttgatcgccgcggggctaacttgggtcaggagctgcgcaacattctctttgctggtactgaccacgaggatatccgcgcgtatgtcaactatgcccatggtgatgagactccccagcagttgtatggtagcgaggggtggcgtcagcagcgtctgcggtccctgaaggccaagtatgaccctacgggcaagttcagctactacgcacctattccttgaagggcgctatataataattacgcccaaagcaatttcccaatgattagtattaatagaaagaattactttgcgttaacttacaagtttcgtattccacatctagagcgccgccacactgtaggcctttagtaaatcagcaaaagtccacgcactgattcatgcatctcgtagcattctgaagagactttttcccatacacgatttgaaaatcatgaacagtagtattacacatcaatcacggcccagtcgatacctcctagtaatcaattactagtccctacctgaatcaagggtcctggacacgcccaacgtcgatcagattacccttacccacataaagcagcatgtaacac

SEQ ID NO: 3 - DNA, 4745 bp, Aspergillus niger
tgtgcaacaaagaatttggttgagcaagcataggtagtccgtgagaatagggcgagcaagtcccgtgcagaggattgcttctgcctgcacggatgacgttggcagcgggcaaacatgcaaggccggaacctgatgggcctcgcgtcatgaccgggggcgatgacatgtttttaagtggtatttagattaacaccttagcgactagaaggaacttttggccccggtgcatacgtggatcccagttcaggatcaagccgggcgcagctaaatgggggccggagaataagggggacacaagatcactgccaaaaacaaagaagcttgctactgacttgtcagccaagtgggtggacaatgggcctagtagggctgaaccccttgataggtcccctgagtttcgttgattttcgggtcggccaggcatggatcggagtccggccaggcagaaagatcctatcaagaagctaaagatgctttttcctggctaagcctctctgggtccaccgatttagtggagcttaaccaatggtttaatcttcattcgtcgactcgcagggctctttttgggtttcgattactcacgtgtttggctccccttaccatcttctcgtatgctggaggagatttctgctgatggttactcagacagatccagcgttccggaattctatgcgactaagcggtccattaatatagcgatttccgaccatttccgaagaatatgagttcagataagggggccttagagacatattctgtgtctaggctttcgtcagagtgaaagcaaactctgcaaacccggtcgggatgctggctaaaagggacaagaaggtttgtcaaagcacccccgcaccagttggcaactgtgaagaacagtagatagtcagtaagccccagactacacgaaaatttctgggcattatgctatttatggcaccggcttatcacaccattccactattacgagggtcaataatctctgccacagtggcttggcagatacgctgggattatcatgctcgggtacagttggacagtgcgcacgagaatggcgcagtcactcagcaacctgaaacaccatagcataagtgtcttggatgtccatgattagtaataggtgcaatgcaccaccctttatgcgtgcaggggtaacttcatagcagttaattaagcttgtggcagccagtgcaatataaactaatgtgatatataatcactgtatactttcctacaataacaagattacactgtagaattatatagattcgatgcttatacaatgagattgcctggcactgaagttctgtgaggtttctagaaattcaggcgctatagggcggattacattgggccggatgaggttgcagtgccagagaaatgttaagtctggttccagtatcagtgagtccgcataattgtcagacagcttcactcaatttcttctctacctgagcggtgttccatgggtcagttcatgtcatatcgtctagtggcacgtcatgacttcactaatggctggctccgcgggcatatattccacctcaatggaaaagagactttccattcggctacacgatagaaattttacgtgacttcataccacaacatagcagtctatgatgctctagatgccgtcactggtgaagtcacagtgcatattccaatcgtccatattaacccttaaacaagaccagttgatctaaacttgtggactagccgtagatctcacgtcctagccaactcccccgacccgatagtatcgacaatgctctcaactataacatctttcctcatccttctgttctttgttaccacagtctccgcctatcccttcgaggagaactcctttcctcgtctccaggatactttagccactggcaccaaacccggtgacccccccaaattcggtggtgggtatgactacgtcgtagtgggcggtggcacaaccgggctcctagtagccagtcggctagcaaaggcaggaaactcagtcgccgttatcgaaaacggcacataccctactgggaatttgactacagtgccagggtacaatgagcagtggtttagcagggtgttgccgtcgaattggacggatatggtgaatgtgtggcctactgtgcagcaggtggtatgttgcccagctctactcgggggtgaatacttacgggcgctaatcctggctttattgcagggagggggagagcagcagcatatgatggggagaatggtaggtagcttattcctctactttatcctgtagcctttgttatggttatgtctgatagaattgcagatcggagggagccagggctttacctttgtcgattacttccgcactactaagggggctatgaaaagatgggctgacgaaactggcgatgattcctggacttgggagaatgtccagcaatactataaaaagtcattcaaattcacaccaccgaacaataccgcaagaccatcgaatgccacaccggactatgagtcaagtcaaatcgttggttctacgcctgggccactagaactcaccttccccaaatatgcccaggcatttggtagctgggttaaacttggtctcaacgaattaaaagctggtatcaatcgagtctttgtcgagggcgatatcaaaggtgccagttggatcttaaatatgatcgattcgcaaacgggcaatcgcgccacaacatatacagctttcttggaaccaacacagaaagatgacacgtcgaagattgacgtgtatgtggagacgctggccgagagaactttgtcaaacactccccctggcagcgaccccgtaaccatcggagtcttagtgaaacggtggggcatgagatttccgatattcgcagggaaagaagtaatcctagctggaggacccatacttactcctcaatttctcatggtgtctggtatcggtcctcaggatcacctgcaggagatgaacattacagtcctagctaatagaccgggagtcggccagaactacaacgatcatattctcttcggcgtcaagcacgcagtacaagtggagacaacatcggtgctcctcaacgataccaggaagtggcaagagtgcgaaagattcaaagctcacgcaaatggcatgctggccgatccgggcccggatttcgccgcctttgtcgattacccggaagatattcgccaaaacctgtctgcccagactaaatcaggtaatttgtggccttgtgtctctcgtggttccgttgtgcctaataatatgattctagatctctcccaattcccctctgactggccagatatcggtatcgtatcttctccactcggcgtcaatggcgacggcaatcacaactatgcagacctcgtctgcatccctatgaagcccatatccaagggaactattaaactcagatctaagtccatggatgataagcctgtgctggatccgcaatggcttaaatctccgacagacatggatactgccgtcgccggtctgcagtaccttctgcgcctctatggaacgaacagcatgaagcccatcttaaatgcctcgggtaaacctatcgatctagaaagttctaacaaggacgacctcattaagtatgtgaaaaacaactatcgaacgttgaatcaccaatctgcatcgtgccgaatgggaaagcgagacgatcctatggccgtggtggatagtaagggcaaggtaatcggagttgatagatgtgagtcctacctacttcgactctggacttgacgtttggttgctcctccgtgttgtacgtacggaactgattgagaaacagtgagaattgctgacccgtccgcctggcctttcttgccagcgggatttccgttaggcactgcctgtgagtaataccctaccattcccacagtcagttttggaatgatcggtctaacctactcaaactgtagatatgtttgcggagaagatagccgataacatcctcagtgatcatggaagtgacaaggatgaggatgaggatgagctgcgagtggaactctagagagagggccctacgtttctcaacggaatgatgaagagtactcattcgaacatgaaaaggcaacgacctactgtaattgcaacagaagtttttaatactgtctcagataatgttaatctactagatcgcactacaggaaaaatgactgcaattggcttgagcgacaatgatccgtttctgaaggactcccaatggcactctcgaacgggccattaatcccaacgaagggagcaagaagaaggtcagagcgaaggctactgagatctgaatatggaaataacatataaattgattaaaagtataattctaatgtagatgttccgttgttgattattttggcatcgagcatattcccgccaggggtaatttagtatcagccacgctatctaatcggtacacgccagtgacatcaaagtacttgccggcgaccgggcatacacaaatcgaatcaaagcctatactagtactctgcagccggcatctactatttccgataaaatcaacgaatctgtttgccagattccaggtaagcaatgattggcatcactttatctgctcccacacactcccgccacatgtctggcctgaagagaagatcattgtcgaaatcgtctctcggaggctaaatatcgaattgccccaccttcccctcatctggtgcagaaagcgaacgtcgttcatgatggttttcacatcaatcctcttaaatcaaatcaggaatcatttttcctcccatggaaa

SEQ ID NO: 4 - DNA, 4145 bp, Aspergillus niger
cctagagtacacgatagtaattgaggaagaaatttatgcgtaagttgtaagagacaattgaacctcgtgaagttggaagaatgcgggtttcagttcgctgacaaccaaaataattaatacatgtcagccaagatagaatgactaaggaataagcgcttgtatgttttgagactagtgatccagcattgaggcctgaggggaacagtcctgttatcgaccattcaatctcaactcagcgactcttgggaattgtcagttgcatattatacaatccaggtagaatatctagtttaacaatcatgtgactctatacccagtttatcagtaccccacctggcaatatactttatcgcaacgtaagaataggcatttgctgggactggacgcagacatagaggataccattgttatgggaatcttcacactgattgataagagaaaatgcgaatggagcgcggcgatctttataactgtgggagctgatcatcacggatatggttaagtttgtccccgctccccaacactggcggcgacccaaatgatgattcgtctttccgcacacctcagacccgaatgagtcagactctctcgtggtaaatgcttgatatcaaattcctgactcttggaccctgactaggtcatttccagcaagtggaaaagggcatagtcccacagggcttcttctctaactcagaagtatctaactgctttaaatgagccatctttcgttgatgctatcttcctttaaactgatcgctatcaattatataggactaactggcattcggtcattatctgcaatgtacctgtcgacttttcttactatctgcttaggccctcaggccgtagcagagatggcatcggcgaccatacaatcttgagcttgatatgcgacaccgacgcaggcgcaaaattcaggagagcaagagttggcgggccaggtcaaagaataaagctgaacataatatggatgagtcgtggttttgctagttctagtactattacgaggtgacatcgtggttaccatgtttcggcttcctctgtcagcactgctgacgaagaatgtatatcatgtatccttaagccgtgccctgtaggtttgctgagtaggtcgtcccttgctccagtaccatctgacataagaataagcatctttgtcttttcttcttcttcttctttttattttttttttttctttatttgctcttttcccctcgcagaaatagatgtgattgtcgagatacactgcgctgtcacagtttatggaatacattgtactgcctaggataggagagatctttacctttgaatgcagccatgattcaactgataaagaaatacaacattacaattcaaacaaggctatgagaggttggcagatgctcattaacatgccgagaagcatagtatctcatcggtccaatgatccggtatgagctgatgtattctcagcagcagatttgaattgcagctgacagcattaaatatactgtaagcgtacccatggcatcggtaatctcccacgggcttctaaaacacctgagtaatgctgaagaaaacccactgaaccattagaaacatgaagcaggggggaaacacgtgcatcctcggtcaatcccaaagttgagacttcgtttcgaggttccttgcagatccccctccgcttagatgaaatgactgtctctgaagagcgtcccccatccgaccgttcatttaacagtctgttttagctcaaacgtgcgtgtcgatcttaaatcgaatacgcaaatacgcaaatactacacggaatgttgcccgtttttgaggatcgatcacaaccgattatacaaaatccattcagcatggaccccctgagcctggcatttaaggtaattaggatgccttatttcaccacccactacacggagcaaagcttttcgtatccaagatgatagctgctgctcttcttctggctgcagtggcccctgtcctggcagccccaaacaccgacgcagtgtctgtttgccagcacctacacacggtttacccccagtacaccgtctgggaccccacaggcacctacgcaactgaaacattctggaatcagtcttactacaacaatgcagtcaaggaatactggaacggcgtgaatgctgacaaccgtccggcctgcgcctttttccctgcaaacgcgcaacatgtctccgtcgcaattcagcagctgaacaaatatcccactgcaccctttgcattgaaaggaggcggtcacaatttcaatgtgggactctcaagtaccaacgggggggtccttatctcgttcaatgagaacctgtcctcaaccacgcgtaactcagacggccagactttcgacgtcggccctggagcccgatggggcgatgtctacgctgtcacggagaagacgaaccaggtcgtcgtaggcggccgattggctaatatcggcgtggcaggattcacgattggagggggattgtcatactactcagctcaatatgtactaccttgccctatccctcctccgccccctttcctctacgtccagcactaacccaactcagggcttatcctgcgacaacgtggtcaagttcgaagtagtcctagcaaacggcaccatcgtcaacgcgaacagcacctctaatccagacctctggtgggccttgcgcggcggcggcaaccgctatggcatcgttaccaagttcacataccagggtcacccactcggcgacaacgggcaagtctggggcggcattcgcttttacagcgccgacaaacgccagcaaatctttgaagctctctccaacttcacaagtgaatacccggacgccaaagccgccgtcatcccaaccttcgactttggcttgcctggagcgatagtctccaacccagccgttttcttcttctacgatggagcgaagcccagcactaacgccttcgtcggactcgacaacatcgaagcccttatcgactctactaagaccaccacctacaccgatctaacgaacgaagcaggcggagccaagatctacggcatcaacgctgccattcgtgtcaacacattccccaacatgccgtcccagcagatgacccaactgcttgaaaaccactggactacctaccagtccatgatcaagaacgattcctccaagaacttggacatccagatcggcacatttaccccgcaaccgctatcagtgcgcattgctcgtgcttccaacaaggccggcggtaatgctctcggcctggaccctgcgaacggtgatcgtgtctggattgagaatgacttgatctgggttaatccggtttgcaatgatgcttgtccggagtatctgcgccaggtgggtgatactgtgaaggaggcatttaataacacgctgttgggaactaagccgacgaattatcaatcgggtgatgtggattggatctcgtacgtgtcctccttcccttcatttctctcttcgtccagtgcagcacgctaacaattatgatagctctaatcctctcttcatgaacgatgccgccgactaccaagacgtatatggcagttatgggccaacgaataaggcgcgactggcgagcattgctaaggcgtatgatccgacgggtttcatgttccgtcagggcggatggtccttttgaacttgactgtgcctgaaatggagcgcaaagcatctacaatttgtttgttgattatttatcgcgtatgatggtctattcagggtgcaaataccccagatacctttagctggataatggaataagataatgttaggattacacattggtcttcagcctatccacctgatcttattgttcagtgatagctccattgaagcttgagctatatatcccccacgaggataggctcgttttacagctagtcagatatcagactacaaactacaagctcaaaatataccagtagtagtaacatacgatacaatacaataaaatctaaccgataacgctagaagagtataaccgacaaaacaaaaagctcccctcccctaagatatcctgctccatcctccccgatagaaaccactacaccccacaatccacacctagaagaacaaatcaaaaacgaaactgacaaccgatatccatacccagtatgaatcccagcataattcttttcaaagacatacacc

SEQ ID NO: 5 - DNA, 4641 bp, Aspergillus niger
gatcgaagaataaaaaggccaagaggcaaccgcaccacaggcatgggattagggaagatgacgaaatcattccccttctctctcgcgcgcccgtataatcaggagagcattgcggcaccggcgacaggtacaaggccggggcgtctgcagttgactgatcaactttcgcagttgaaattggaaagttttctgataagatgcggtgtttgcactgtttgtttccccaccgtctgccgctcggattgattccgcgacggagcagctcaacaccgtatgaacaccgtttacaacactggaaatatcccgattaagtctttaaaaagaggcttcgagaggattacagataattacagatgcccccatattgaaaaaattttcagtcactttagtcttactgtcagctgaagctttgcggcggtaacaacgacggcgttgccagcaggacggtctgtatgtgcgactaccatgaggagtataccggtttagcgccgggaatccaaaccaaggacacagacacctagaaaatcatacccgctacaggtgctggggtggcttgcaagctcggataaaacgaagaatggccgataaggctgaattcaaccaggtctctttctaggattggttggacgattaccggcacaaaaaggtgttgatgacaagagtttcaatctgcctcatcgaaggcagatcccaacttttgcattggaatctctgtacaggtacgatgccatatagacttcatccatgttatgaattctaatctcagccactacgcctagtctatctcaaggaaaagtacaagtatattactcaacaagctgggtaacaccatcgaaaacaggatggacctgggaagggaagggagtgattcacctccataatcttttactacatcaagttgctactaaaccaatcgatatgctcgactgcgcttgactgctaccgtcacggatttacatctgcaatatgtcgccgaggtatctccggcttcgtgtctaataggtggtaatctattagcagccttgctatcagcatccagccacagaacacacggaaatttgccaaataaggccaattcgggatagacaggctagaacggtgattaattccgagtccaaacaccagattggattaccaagtggagaatcgtagaatgaagcggttgggagcaaacagaaatgacggacctgatgagagggcgtgagctggctgaacttcggaggtgcgacggtgatggtggaacccgtggaggggtatgaatgcaggacgagacgaagcgcaagccaacaccttctttctgagtaaattgccatgaccctgtcaattccgtgattaggcaagtgctttgcgccttcattggcccttatccctgcatccgcgggcattggtagggaacatcttcagttttgggctctgtgtttaacctgcgtgcacaccttactgtgccttgaagctactcaataagattttctcattttcgactcttttatgtcacagatactggcaaggcggctgtctagttctccgggttggtttcccgtccgctcgattgacaacgctaaaatctagtacgctgccaactagctttcccactatataagggagctccactgcgaataaagtgtgcctgtcatcaagagacccactaaccgggccacccaaccgtcaaatcaagcctcggtttgttattagacaatctggaccaccggtgcaatcgccagtttgggcttgcagcatgtcttactacgcagacggcaattgatcgacacgctcgtatagggattcgcacacgaacgccttcgcttcatactatctgctccacagaataactttaccctctccgtctctggctaccccaattcaatcgtgccatacgggttgacttgccccgtgtagccatgtacttactacttacttcgttgcgactcgggaccactccggctgtcaaagcacaggcaaggagtatccgctatgaaaaggcgctaatcgtggaagtcttcccgcagcccgcttgatctaggggcttcggcatatattatgaagtgtagccatctccagggactctgctaacggagaaattctatcagcgttggactcgaaatgaaggtctccagtgcggcagtccctgttctcagcattctgcccgaggccagcctcgggcttggctctacgcaactttgtgtatgtggttcatgttcagtacttagtcggtgtactaatagccttgtcacctagtgcgccgctctgcaagcgacctccctgcgtaatcaagtcctttaccctaccagcgctgcatacaatgagtccgtggcatcttactttgcggtcaatgttcagctcgaccctacgtgtattgtacagccacactctacagaggatgtctcgctcattgtctcgactctgacccaaaccggcgaaacacaatgcccatttgccgtgcgcagtggtggtcataccacctggcctggtgcagcggatatcggacagggtgttaccattgatttgtccatgatgaacagcaccacataccacaaggacaaaggcgttgcatctatccagccgggtgcgcgctggcaggcggtctacaaggctcttgatccgtacggagtcacagtacctggcggacgcggcggaccagttggcgtgggtgggttcttgatcggaggtaagattcagcgacagttcaaacaaaagtggttgcatgtctaatgccctgataggtggaaacaccttctacaccgctcgtgtaggcttcgcatgtgataatatcgagaactttgaagtcgttcttgccagtggatcgatcgtcaatgccaaccggactagccatcctgatctgtacaaggcgctgaagggtggctcgatcaactttggagttgttacaaagtatgacctcaagacgttgcctcatgatatgctttggggtggactggtcgtctatgataattcaaccaccgcccggcagctctcggccgctgtgaactttaccaacaacatccacaatgacccatacgcatcctggattgggatgtgggaatacagctcgacaacgggacagaacatcattgccgatgccttggaatacaccaagcctgttgcctttgctcctgcattccatgaatttacgagtattccaaacaccaccgacacgatgcgttttgctacgatttacaacctgacacaagagctggttcaggcggcgggctaccggtaagtctccaatagatagccatcttcggtcctctttcttcttggaaggcagtcgatggtggtgtttaagtctggttctcgttgttcggggcgctaacaactcagtgatgttttcaccaccggcacgtacaagaacgacgtcaaagtccttcgcaaggcgatcgaccttcacaacaacaacattgaaaaggccaaggctcatgtcaagagtgactactgggccatggataccatcatccagccctggcccaagcttttcgctcagcacagcgtggagaagggtggcaacgttctgggtttggaacggtttgacgagaacctgatccgtatgtaacccgaccttgtttctagcgcagcactaatccgtttacagaaatactattcgactactcctgggatgatgcaagggacgacgatctcttcatcggccttggccagtcgatcctggaggaggttggtgaatacgccaaatcgattggggcgcacaatgagtacatctatctcaattatgccgacaagtcgcagaaccctctcaagggctatggcgaggagaatgttgagttcatctcccgcgtggccaagcagtacgatccagatggagtgtttcaggtacaggttccaggtggcttcaagatcagccaggtttaagcaccatcgagtccaaaatttacgactagtcgaagtaattttgtacagtagactacgaaaaagaacagaatagattagacaacacaagctccaccccctcattttgccttgctaggctgcgccatttttgacacggaattagtgcctttttaaatttgcggcgcagtcaacccgcatcgcggtggagccgataatgaagccagcacgagccgtttcaacgcgatccgaactagcacggtctaatcgttcgcctcttggggctctaccaccgatcctttccactcctcccgccgtctctcgggcctctggacggccaggctttggcccggacgacggctagacgcgatggcatattggacgctccacaaggatgacactatccatgcaccgggaaaattcttaagcttggttggattgttattgttaatcacagtaatatacggaatgtggtacaatttgccacggctaacaaaggatgcgcatatccttgtcatcttgggagagcgtgcataagctttcgatagcaccttccgtgttggaatccatcgagggtaatcaatacgcttttccatgaagagaaaaagcaagaaaaaaacaaaaaatgaacaaatgcaaattctttctccccatcatgtctgtgccatcgactaggccttcccatgcctcaggggccacctcagagtataagctcccttctggaacccttctatgctttatgacagcgtgaggcttcagtgagaggctgttggccttcgatgccacttgaatatggccattactaca

SEQ ID NO: 6 - DNA, 3837 bp, Aspergillus niger
aagacaatacggagtacattgacctttattattacttgatactatcgaaggaagagaataaatactactccttctgaatgtgcatagggttatgtatatgctaaaagacaggttatcactagcgtgcatcgaatctgactgatcagttgcagttcctggttgaacacgcacatatttccttctaattgattggatctattcagcttcttctatgcgaaggcggtccaagattgattggtctcggccactgggtaagctaaggctgtcacttcaggcaggggtaggcctgagactgcaggatggtggccagagccaatttctgaaaccctgtagatccgcaacatctcccaaataaagctattgtaatcacatggctcaatcttagaaggaatacagtactacgaatggcggttgattgcaagcttcattctacctgctgcggtgaacaacaccccacaacaaccatcttgacaaataatacgtcgtcatcaaaaaggagaagctaccaaatcaggtcatcttcggcgtatgagaatctgacaatgtcctcttcagctgaatgtcggccgattgggttggggggtgtgggaggcggatcccaatacgagtcccacccgtgcgtctaccaaaatggttcgctgatcctcgctcgctccatcgtcagcgatgtgcttgtggactatgtaatgcccgacgaacgatgcaatgggtaatttcagctccgccgcgtattttggatagacgatcaatccaggtattgattaaatgttcgcccgagccccgatttaaagcttcaaatgactcccaaccacaagcttgcgacaaccggatgacttactgaacttatcagccagtattcaatctgacggtcaggatggtcaatcttgcctatattctgggtgccgttgcggttttttcaacatcgggaaccgctgcagacgtttctctatcttcctctgttgctgcttccacgaccccatcagctgccggttctctttcctcgctgggcctttctctcccagccggcaatgtgctcgtcgggaatgtggggtacacctgcaatctgctgagtcgtacatttccaaagaatgagacctttaccgccagctcaccctactatgaggctctaattgatgagacctggtcagggaacagccgcctgaacgcgtcctgcattgtgacgcccagatctgctcaggaggtttcgcttgtcatccaaatccttagtattctggagaccaaattctccattcgctcgggtgggcacagttccaatccagggttcagttcgattggtagcaacggagtgctagttgcgcttggaaggttgaacacactctcgatcagcgcggaccggaagaccctcactgttggaccgggcaatcgctgggaagccgtgtaccaatatctggagcagtacaatctgaccgtactcggggggcgagagcccgttgtgggagttggtggcttcgtcctggggggtaagccaatgcttcctctatcatattctgggaaaataaacatgatactaattacaataggtggtcttagtctcttctataacaccaatggtctggccattgacacggtcacccgattccaggtggtaacccccaatgggaccatcgtcaacgctacccaaaccgagcatgccgatctatacaaggggctgaaaggaggtctgaacaactttggtaggcctaatactcacctcggtgacctcttttatgtgactaatcgaacccaggcatcgtggtggaatacgacctcaccaccaataccggcatcgatgtctggttcgaggtcaagaactataccctcgctgagactcccgcactgcttgctgcctacgcagcatatctccaaaatgccgatatccgcagcaatgtcgagattcagacgaatccagcatatacactagtcttctacggatatctcgaccatgtgtcagcgccatccgcctttaatgctttctcccaagtcccgtcggtctcgactgtttatcctcctaccaatgcctccctaaatcaagtcctcctcgacattggcaatgccggtgtggttggctcattatatacctatagcatttcttttgccttcaaggttaccagccccagctttctccaggaaagcttcaaggcctaccttaaaactgccgcatcgctcctgcctttgggcgtaatactcgagtacgtgcctcagggcatcatcccaaacctagttaccaaaagccagacccagaatggtggtaatttgtttggcctggaggccacacctcaagtttgtatgtatcccttgtgcccgatcaattcccaatctatctgaggaacgcagtactaactgaatacttggacagggggggagatctttgcccaattccccgccacggttagtcagtcaacggtagccaacgccgtggaatctcttctagccaaccttacctcgagcgcccagtctcagagtgttcatcttccttacattttcgccaatgatgctggccctaatcagcaggtcctgcgaggatatggtgaagacaatgtcaagtatattgccgccgttgctgagaggtatgatccaaggggcgtgatgcagaagttgcagaatgatgcctatttcgtgtcgaaagagttgtaacgggttatcactgtatcaatatctcgtctattttcgggatcgtctgtacctacagcatactctgtaataattttaataaggggtgatccttctgcttccataccaccggtcgtcacttcttgggcgcaaatgacgagggagacgggaggtttgacctcagtctgggagtcagcaaatactacaagtagtcatgcgttaatgtctcaattatcgagcctccatgttgggttttaggtgacggtgaaagctgatacactgcaagacgctgaaaggacatgatgttacaatgatgctatgaaggtgggtgtgagtggtggtactggattacgcaaaaatatatattcagactatgaggctcagggtcattggcaggtttcacccatcgacatatatttctacgcccatctatttgatggcagagtctgctgtaaacaaaagtttcttttgccttaagaaggttcaatcagaaatttccaggagttttatatattaagcagagcgaaatcaaatgtggaaaaaaaaagacaatcaaaaaggaatggggcaagccgggcttgaaccggccacctccagatcttcagtctgacgcgctcccagatgcgccattgccccggtgattacgtcatatgctacacatagctatataaatattaaagctactgattaatattttgcttgcttaacgctctaaaggcagcatgaaacctaatattaactaactcgatattatataccaataattcatgaacctcaaaaatgtgcactatgctatttacgctcaatgattcaatgctatcacctctcctgctcaagccgcctgctgaactccatggatgatattcatgaactcgctgccatatattgctcacccgatcccgttgatttcgcgtgaaccacaagtgtatgctttcatgtctgccttttaataaatcaccccatactaccttcatgtaaaagccgctcctatattttccattgggtgaaatgatcgcacctcttcgtatagttatggcccgcgggtctcctactcgactcgatgatctaactccatattcatactacataggctaccacatcatcgctcacagctggaattgacttgacaaatatccccatctcggcagctggccgtataggactggcgattatgtacttaaattgagaaagtaaatacaactagagagcacgctcactctaatccca

SEQ ID NO: 7 - DNA, 1713 bp, Aspergillus niger
  CDS (1)..(1713)
atgaaggcttcctggtcttttgcagcatctgtcgctgctctcgcctccaaggccgtcgcttctagcgactgccactgcctgcccggcgatagctgctggccctcgacctccagctgggactccctcaacaacactgtgggcggtcgcttggttgcgactgttcccattggtactccttgccatgaccctaactacaatggcgccgagtgcacgaacctgcaggatgactggtactacccgcagactcacttggtctcttcttcatctgttatgcaaccgtactttgccaaccagagctgtgatcctttccagcccgagtcccggccctgcctgcttggcaactacgtgagctacgccgtcaacgtctccacgaccgatgatgttgttgctgcggtcaagttcgcccaggagaacaacatccgtctcgtgatcaggaacaccggtcatgactaccttggccgttccaccggtgctggtgccctggccatctggactcactacctgaacgacgtcgaaatcacagagtggtcggattccacctacgcgggttcggctgtcaagctgggcagtggtgtcacgggctacaatgtgctcgatgctactcacggaaagggaattgttgtcgtcggcggcgaatgccctactgtcggcctcgctggtggatacaccatgggcggcggtcactccgccctgagcactgccttcggtctgggtgctgaccagaccctgtccttcgaggttgtcaccgcttcgggcgaggtcatcacggcctcccggaccaacaacaccgatctgtactgggccctcagtggtggtggtgccggtaacttcggtgttgtcacctctctcaccgtcaaggcccatcccgatgccaccatctccggtgctgcgctcgaattcaccatcgccaacatcacctccgatctcttctacgaggctgtggagcgcttccacaccttgctgcccgccatggttgatgccggcaccaccgtcatctatgagatgaccaaccaggtcttcctcatcaaccccttgaccgcctacaacaagaccaccgctgaggtcaagaccatcttgtctcccttcctctctgctctgaccgacctgggcatcgagtacaccgtcgcttacacccagtactcctcttactacgaccactacgagaagtacatgggacccctcccctacggcaacctcgaggtcggccagtacaactacggcggccgcctcctcccccgtgacacccttacctcgaacgccgccgatctcgtctccgtcctccgcaacatcacctccgacggtctcatcgccgtcggcgtcggcctgaacgtcaccaactccaacgacaccgccaacgctgtcttccagccctggcgtaacgccgccgtgaccatgcagttcggttccacctggaacgagactgccccttggtccgagatggtcgccgaccagctgcgcattgcccacgactacattccccagttcgaggccgtgacccctggctcgggcgcctacgagaacgagggcagcttccgtcagcagaactggcagaaggaattcttcggcgataactacgcccagctctgcgaggtcaaggagaagtatgatcccgaccatgtcttctacgtcaccaagggtgccggtagcgagtactggtctgtggccgagtcgggtcgcatgtgcaagacccagcaggcgtgcgctgctgtttga

SEQ ID NO: 8 - DNA, 1503 bp, Aspergillus niger
  CDS (1)..(1503)
atgtttcgactcaagatggaggtgctcacggccatcgcggcctgggcattcgccacggtctcgcagcaaatccctcgctcctccagctgggaaagtgacctccagaccctctcgggtaggctttccaatacctcccagatctattatccgggctccagtggattcacaaatgccaccacgcggtggtccgtcctggatgagcctgaggttaatgttgtcgtggtccctggcaccgaaaatgatgtcgccgaaattgtgaaatttgccaatcagaaggatgtccccttcctaacctacaatggtgtgcacggtgccctcatctctctgggagaaatgacccatggtattgctatctacatgggccagttgagtagtgtcgaggtcgcagctgacggcaagactgccacgatcggaggtggaaccatgtccaaggaggtcaccgaccagctttgggccgcaggaaagcagaccgtgactggaacctgtgaatgtgtcagtcttgtcggtcctgcgcttggtggtggccatggttggctccagggccaccacggccttgttcttgatcagtttgtctcgatgaacatcgtgctggcgaacggcactctgacccacatcgacgccaactcggacctctggtgggccgtcaagggtgccggccacaactttggcatcgtcacttccctcaccatgaagatctatgacatcgagtacagcgattgggccatcgagacgctgaccttcagtggcgacaaggttgccgaggtctaccaggctgccaatgactacctggtcaagaacggcacccaggctgccggcgtgatcaactggtcgtactggatgaacaatgcggatgccgaccccaacaacccggtcatcatcttctacatcatccaggagggagtgaagaccgtcgattccgtctacaccgcgcctttccgcaagatcggtcccatttctgtgtcccccaacaacggcacatacaaggatctcgccgcatggactgaggttgctgtcgactccgccccctgccagaagatgggtatggccaacccccgttacccgatctacctcgaaacgtacaacgtcactgcccagcagaaggcatgggacgtctacgcgaatgctacccgtggcttttcggcgttcaacaactccatcttcatgttcgagggatactcagttggcggcgtgcacgatgttgatagccggtcgagcgcttttgccttccgtaatgaaaacgtgctggctgctcccatgatcaactactaccctgatggcgccgaacttgatcgccgcggggctaacttgggtcaggagctgcgcaacattctctttgctggtactgaccacgaggatatccgcgcgtatgtcaactatgcccatggtgatgagactccccagcagttgtatggtagcgaggggtggcgtcagcagcgtctgcggtccctgaaggccaagtatgaccctacgggcaagttcagctactacgcacctattccttga

SEQ ID NO: 9 - DNA, 1995 bp, Aspergillus niger
  CDS (1)..(1995)
atgctctcaactataacatctttcctcatccttctgttctttgttaccacagtctccgcctatcccttcgaggagaactcctttcctcgtctccaggatactttagccactggcaccaaacccggtgacccccccaaattcggtggtgggtatgactacgtcgtagtgggcggtggcacaaccgggctcctagtagccagtcggctagcaaaggcaggaaactcagtcgccgttatcgaaaacggcacataccctactgggaatttgactacagtgccagggtacaatgagcagtggtttagcagggtgttgccgtcgaattggacggatatggtgaatgtgtggcctactgtgcagcaggtgggagggggagagcagcagcatatgatggggagaatgcctttgttatggttatgtctgatagaattgcagatcggagggagccagggctttacctttgtcgattacttccgcactactaagggggctatgaaaagatgggctgacgaaactggcgatgattcctggacttgggagaatgtccagcaatactataaaaagtcattcaaattcacaccaccgaacaataccgcaagaccatcgaatgccacaccggactatgagtcaagtcaaatcgttggttctacgcctgggccactagaactcaccttccccaaatatgcccaggcatttggtagctgggttaaacttggtctcaacgaattaaaagctggtatcaatcgagtctttgtcgagggcgatatcaaaggtgccagttggatcttaaatatgatcgattcgcaaacgggcaatcgcgccacaacatatacagctttcttggaaccaacacagaaagatgacacgtcgaagattgacgtgtatgtggagacgctggccgagagaactttgtcaaacactccccctggcagcgaccccgtaaccatcggagtcttagtgaaacggtggggcatgagatttccgatattcgcagggaaagaagtaatcctagctggaggacccatacttactcctcaatttctcatggtgtctggtatcggtcctcaggatcacctgcaggagatgaacattacagtcctagctaatagaccgggagtcggccagaactacaacgatcatattctcttcggcgtcaagcacgcagtacaagtggagacaacatcggtgctcctcaacgataccaggaagtggcaagagtgcgaaagattcaaagctcacgcaaatggcatgctggccgatccgggcccggatttcgccgcctttgtcgattacccggaagatattcgccaaaacctgtctgcccagactaaatcaggtaatttgtggccttgtgtctctcgtggttccgttgtgcctaataatatgattctagatctctcccaattcccctctgactggccagatatcggtatcgtatcttctccactcggcgtcaatggcgacggcaatcacaactatgcagacctcgtctgcatccctatgaagcccatatccaagggaactattaaactcagatctaagtccatggatgataagcctgtgctggatccgcaatggcttaaatctccgacagacatggatactgccgtcgccggtctgcagtaccttctgcgcctctatggaacgaacagcatgaagcccatcttaaatgcctcgggtaaacctatcgatctagaaagttctaacaaggacgacctcattaagtatgtgaaaaacaactatcgaacgttgaatcaccaatctgcatcgtgccgaatgggaaagcgagacgatcctatggccgtggtggatagtaagggcaaggtaatcggagttgatagattgagaattgctgacccgtccgcctggcctttcttgccagcgggatttccgttaggcactgcctatatgtttgcggagaagatagccgataacatcctcagtgatcatggaagtgacaaggatgaggatgaggatgagctgcgagtggaactctag

SEQ ID NO: 10 - DNA, 1578 bp, Aspergillus niger
  CDS (1)..(1578)
atgatagctgctgctcttcttctggctgcagtggcccctgtcctggcagccccaaacaccgacgcagtgtctgtttgccagcacctacacacggtttacccccagtacaccgtctgggaccccacaggcacctacgcaactgaaacattctggaatcagtcttactacaacaatgcagtcaaggaatactggaacggcgtgaatgctgacaaccgtccggcctgcgcctttttccctgcaaacgcgcaacatgtctccgtcgcaattcagcagctgaacaaatatcccactgcaccctttgcattgaaaggaggcggtcacaatttcaatgtgggactctcaagtaccaacgggggggtccttatctcgttcaatgagaacctgtcctcaaccacgcgtaactcagacggccagactttcgacgtcggccctggagcccgatggggcgatgtctacgctgtcacggagaagacgaaccaggtcgtcgtaggcggccgattggctaatatcggcgtggcaggattcacgattggagggggattgtcatactactcagctcaatatggcttatcctgcgacaacgtggtcaagttcgaagtagtcctagcaaacggcaccatcgtcaacgcgaacagcacctctaatccagacctctggtgggccttgcgcggcggcggcaaccgctatggcatcgttaccaagttcacataccagggtcacccactcggcgacaacgggcaagtctggggcggcattcgcttttacagcgccgacaaacgccagcaaatctttgaagctctctccaacttcacaagtgaatacccggacgccaaagccgccgtcatcccaaccttcgactttggcttgcctggagcgatagtctccaacccagccgttttcttcttctacgatggagcgaagcccagcactaacgccttcgtcggactcgacaacatcgaagcccttatcgactctactaagaccaccacctacaccgatctaacgaacgaagcaggcggagccaagatctacggcatcaacgctgccattcgtgtcaacacattccccaacatgccgtcccagcagatgacccaactgcttgaaaaccactggactacctaccagtccatgatcaagaacgattcctccaagaacttggacatccagatcggcacatttaccccgcaaccgctatcagtgcgcattgctcgtgcttccaacaaggccggcggtaatgctctcggcctggaccctgcgaacggtgatcgtgtctggattgagaatgacttgatctgggttaatccggtttgcaatgatgcttgtccggagtatctgcgccaggtgggtgatactgtgaaggaggcatttaataacacgctgttgggaactaagccgacgaattatcaatcgggtgatgtggattggatctcctctaatcctctcttcatgaacgatgccgccgactaccaagacgtatatggcagttatgggccaacgaataaggcgcgactggcgagcattgctaaggcgtatgatccgacgggtttcatgttccgtcagggcggatggtccttttga

SEQ ID NO: 11 - DNA, 1656 bp, Aspergillus niger
  CDS (1)..(1656)
atgaaggtctccagtgcggcagtccctgttctcagcattctgcccgaggccagcctcgggcttggctctacgcaactttgttgcgccgctctgcaagcgacctccctgcgtaatcaagtcctttaccctaccagcgctgcatacaatgagtccgtggcatcttactttgcggtcaatgttcagctcgaccctacgtgtattgtacagccacactctacagaggatgtctcgctcattgtctcgactctgacccaaaccggcgaaacacaatgcccatttgccgtgcgcagtggtggtcataccacctggcctggtgcagcggatatcggacagggtgttaccattgatttgtccatgatgaacagcaccacataccacaaggacaaaggcgttgcatctatccagccgggtgcgcgctggcaggcggtctacaaggctcttgatccgtacggagtcacagtacctggcggacgcggcggaccagttggcgtgggtgggttcttgatcggaggtggaaacaccttctacaccgctcgtgtaggcttcgcatgtgataatatcgagaactttgaagtcgttcttgccagtggatcgatcgtcaatgccaaccggactagccatcctgatctgtacaaggcgctgaagggtggctcgatcaactttggagttgttacaaagtatgacctcaagacgttgcctcatgatatgctttggggtggactggtcgtctatgataattcaaccaccgcccggcagctctcggccgctgtgaactttaccaacaacatccacaatgacccatacgcatcctggattgggatgtgggaatacagctcgacaacgggacagaacatcattgccgatgccttggaatacaccaagcctgttgcctttgctcctgcattccatgaatttacgagtattccaaacaccaccgacacgatgcgttttgctacgatttacaacctgacacaagagctggttcaggcggcgggctaccgtgatgttttcaccaccggcacgtacaagaacgacgtcaaagtccttcgcaaggcgatcgaccttcacaacaacaacattgaaaaggccaaggctcatgtcaagagtgactactgggccatggataccatcatccagccctggcccaagcttttcgctcagcacagcgtggagaagggtggcaacgttctgggtttggaacggtttgacgagaacctgatccaaatactattcgactactcctgggatgatgcaagggacgacgatctcttcatcggccttggccagtcgatcctggaggaggttggtgaatacgccaaatcgattggggcgcacaatgagtacatctatctcaattatgccgacaagtcgcagaaccctctcaagggctatggcgaggagaatgttgagttcatctcccgcgtggccaagcagtacgatccagatggagtgtttcagtgcctttttaaatttgcggcgcagtcaacccgcatcgcggtggagccgataatgaagccagcacgagccgtttcaacgcgatccgaactagcacggtctaatcgttcgcctcttggggctctaccaccgatcctttccactcctcccgccgtctctcgggcctctggacggccaggctttggcccggacgacggctag

SEQ ID NO: 12 - DNA, 1662 bp, Aspergillus niger
  CDS (1)..(1662)
atggtcaatcttgcctatattctgggtgccgttgcggttttttcaacatcgggaaccgctgcagacgtttctctatcttcctctgttgctgcttccacgaccccatcagctgccggttctctttcctcgctgggcctttctctcccagccggcaatgtgctcgtcgggaatgtggggtacacctgcaatctgctgagtcgtacatttccaaagaatgagacctttaccgccagctcaccctactatgaggctctaattgatgagacctggtcagggaacagccgcctgaacgcgtcctgcattgtgacgcccagatctgctcaggaggtttcgcttgtcatccaaatccttagtattctggagaccaaattctccattcgctcgggtgggcacagttccaatccagggttcagttcgattggtagcaacggagtgctagttgcgcttggaaggttgaacacactctcgatcagcgcggaccggaagaccctcactgttggaccgggcaatcgctgggaagccgtgtaccaatatctggagcagtacaatctgaccgtactcggggggcgagagcccgttgtgggagttggtggcttcgtcctggggggtggtcttagtctcttctataacaccaatggtctggccattgacacggtcacccgattccaggtggtaacccccaatgggaccatcgtcaacgctacccaaaccgagcatgccgatctatacaaggggctgaaaggaggtctgaacaactttggcatcgtggtggaatacgacctcaccaccaataccggcatcgatgtctggttcgaggtcaagaactataccctcgctgagactcccgcactgcttgctgcctacgcagcatatctccaaaatgccgatatccgcagcaatgtcgagattcagacgaatccagcatatacactagtcttctacggatatctcgaccatgtgtcagcgccatccgcctttaatgctttctcccaagtcccgtcggtctcgactgtttatcctcctaccaatgcctccctaaatcaagtcctcctcgacattggcaatgccggtgtggttggctcattatatacctatagcatttcttttgccttcaaggttaccagccccagctttctccaggaaagcttcaaggcctaccttaaaactgccgcatcgctcctgcctttgggcgtaatactcgagtacgtgcctcagggcatcatcccaaacctagttaccaaaagccagacccagaatggtggtaatttgtttggcctggaggccacacctcaagtttggggggagatctttgcccaattccccgccacggttagtcagtcaacggtagccaacgccgtggaatctcttctagccaaccttacctcgagcgcccagtctcagagtgttcatcttccttacattttcgccaatgatgctggccctaatcagcaggtcctgcgaggatatggtgaagacaatgtcaagtatattgccgccgttgctgagaggggtgatccttctgcttccataccaccggtcgtcacttcttgggcgcaaatgacgagggagacgggaggtttgacctcagtctgggagtcagcaaatactacaagtagtcatgcgttaatgtctcaattatcgagcctccatgttgggttttag

SEQ ID NO: 13 - protein, 570 aa, Aspergillus niger
MKASWSFAASVAALASKAVASSDCHCLPGDSCWPSTSSWDSLNNTVGGRLVATVPIGTPCHDPNYNGAECTNLQDDWYYPQTHLVSSSSVMQPYFANQSCDPFQPESRPCLLGNYVSYAVNVSTTDDVVAAVKFAQENNIRLVIRNTGHDYLGRSTGAGALAIWTHYLNDVEITEWSDSTYAGSAVKLGSGVTGYNVLDATHGKGIVVVGGECPTVGLAGGYTMGGGHSALSTAFGLGADQTLSFEVVTASGEVITASRTNNTDLYWALSGGGAGNFGVVTSLTVKAHPDATISGAALEFTIANITSDLFYEAVERFHTLLPAMVDAGTTVIYEMTNQVFLINPLTAYNKTTAEVKTILSPFLSALTDLGIEYTVAYTQYSSYYDHYEKYMGPLPYGNLEVGQYNYGGRLLPRDTLTSNAADLVSVLRNITSDGLIAVGVGLNVTNSNDTANAVFQPWRNAAVTMQFGSTWNETAPWSEMVADQLRIAHDYIPQFEAVTPGSGAYENEGSFRQQNWQKEFFGDNYAQLCEVKEKYDPDHVFYVTKGAGSEYWSVAESGRMCKTQQACAAV

SEQ ID NO: 14 - protein, 500 aa, Aspergillus niger
MFRLKMEVLTAIAAWAFATVSQQIPRSSSWESDLQTLSGRLSNTSQIYYPGSSGFTNATTRWSVLDEPEVNVVVVPGTENDVAEIVKFANQKDVPFLTYNGVHGALISLGEMTHGIAIYMGQLSSVEVAADGKTATIGGGTMSKEVTDQLWAAGKQTVTGTCECVSLVGPALGGGHGWLQGHHGLVLDQFVSMNIVLANGTLTHIDANSDLWWAVKGAGHNFGIVTSLTMKIYDIEYSDWAIETLTFSGDKVAEVYQAANDYLVKNGTQAAGVINWSYWMNNADADPNNPVIIFYIIQEGVKTVDSVYTAPFRKIGPISVSPNNGTYKDLAAWTEVAVDSAPCQKMGMANPRYPIYLETYNVTAQQKAWDVYANATRGFSAFNNSIFMFEGYSVGGVHDVDSRSSAFAFRNENVLAAPMINYYPDGAELDRRGANLGQELRNILFAGTDHEDIRAYVNYAHGDETPQQLYGSEGWRQQRLRSLKAKYDPTGKFSYYAPIP

SEQ ID NO: 15 - protein, 664 aa, Aspergillus niger
MLSTITSFLILLFFVTTVSAYPFEENSFPRLQDTLATGTKPGDPPKFGGGYDYVVVGGGTTGLLVASRLAKAGNSVAVIENGTYPTGNLTTVPGYNEQWFSRVLPSNWTDMVNVWPTVQQVGGGEQQHMMGRMPLLWLCLIELQIGGSQGFTFVDYFRTTKGAMKRWADETGDDSWTWENVQQYYKKSFKFTPPNNTARPSNATPDYESSQIVGSTPGPLELTFPKYAQAFGSWVKLGLNELKAGINRVFVEGDIKGASWILNMIDSQTGNRATTYTAFLEPTQKDDTSKIDVYVETLAERTLSNTPPGSDPVTIGVLVKRWGMRFPIFAGKEVILAGGPILTPQFLMVSGIGPQDHLQEMNITVLANRPGVGQNYNDHILFGVKHAVQVETTSVLLNDTRKWQECERFKAHANGMLADPGPDFAAFVDYPEDIRQNLSAQTKSGNLWPCVSRGSVVPNNMILDLSQFPSDWPDIGIVSSPLGVNGDGNHNYADLVCIPMKPISKGTIKLRSKSMDDKPVLDPQWLKSPTDMDTAVAGLQYLLRLYGTNSMKPILNASGKPIDLESSNKDDLIKYVKNNYRTLNHQSASCRMGKRDDPMAVVDSKGKVIGVDRLRIADPSAWPFLPAGFPLGTAYMFAEKIADNILSDHGSDKDEDEDELRVEL

SEQ ID NO: 16 - protein, 525 aa, Aspergillus niger
MIAAALLLAAVAPVLAAPNTDAVSVCQHLHTVYPQYTVWDPTGTYATETFWNQSYYNNAVKEYWNGVNADNRPACAFFPANAQHVSVAIQQLNKYPTAPFALKGGGHNFNVGLSSTNGGVLISFNENLSSTTRNSDGQTFDVGPGARWGDVYAVTEKTNQVVVGGRLANIGVAGFTIGGGLSYYSAQYGLSCDNVVKFEVVLANGTIVNANSTSNPDLWWALRGGGNRYGIVTKFTYQGHPLGDNGQVWGGIRFYSADKRQQIFEALSNFTSEYPDAKAAVIPTFDFGLPGAIVSNPAVFFFYDGAKPSTNAFVGLDNIEALIDSTKTTTYTDLTNEAGGAKIYGINAAIRVNTFPNMPSQQMTQLLENHWTTYQSMIKNDSSKNLDIQIGTFTPQPLSVRIARASNKAGGNALGLDPANGDRVWIENDLIWVNPVCNDACPEYLRQVGDTVKEAFNNTLLGTKPTNYQSGDVDWISSNPLFMNDAADYQDVYGSYGPTNKARLASIAKAYDPTGFMFRQGGWSF

SEQ ID NO: 17 - protein, 551 aa, Aspergillus niger
MKVSSAAVPVLSILPEASLGLGSTQLCCAALQATSLRNQVLYPTSAAYNESVASYFAVNVQLDPTCIVQPHSTEDVSLIVSTLTQTGETQCPFAVRSGGHTTWPGAADIGQGVTIDLSMMNSTTYHKDKGVASIQPGARWQAVYKALDPYGVTVPGGRGGPVGVGGFLIGGGNTFYTARVGFACDNIENFEVVLASGSIVNANRTSHPDLYKALKGGSINFGVVTKYDLKTLPHDMLWGGLVVYDNSTTARQLSAAVNFTNNIHNDPYASWIGMWEYSSTTGQNIIADALEYTKPVAFAPAFHEFTSIPNTTDTMRFATIYNLTQELVQAAGYRDVFTTGTYKNDVKVLRKAIDLHNNNIEKAKAHVKSDYWAMDTIIQPWPKLFAQHSVEKGGNVLGLERFDENLIQILFDYSWDDARDDDLFIGLGQSILEEVGEYAKSIGAHNEYIYLNYADKSQNPLKGYGEENVEFISRVAKQYDPDGVFQCLFKFAAQSTRIAVEPIMKPARAVSTRSELARSNRSPLGALPPILSTPPAVSRASGRPGFGPDDG

SEQ ID NO: 18 - protein, 553 aa, Aspergillus niger
MVNLAYILGAVAVFSTSGTAADVSLSSSVAASTTPSAAGSLSSLGLSLPAGNVLVGNVGYTCNLLSRTFPKNETFTASSPYYEALIDETWSGNSRLNASCIVTPRSAQEVSLVIQILSILETKFSIRSGGHSSNPGFSSIGSNGVLVALGRLNTLSISADRKTLTVGPGNRWEAVYQYLEQYNLTVLGGREPVVGVGGFVLGGGLSLFYNTNGLAIDTVTRFQVVTPNGTIVNATQTEHADLYKGLKGGLNNFGIVVEYDLTTNTGIDVWFEVKNYTLAETPALLAAYAAYLQNADIRSNVEIQTNPAYTLVFYGYLDHVSAPSAFNAFSQVPSVSTVYPPTNASLNQVLLDIGNAGVVGSLYTYSISFAFKVTSPSFLQESFKAYLKTAASLLPLGVILEYVPQGIIPNLVTKSQTQNGGNLFGLEATPQVWGEIFAQFPATVSQSTVANAVESLLANLTSSAQSQSVHLPYIFANDAGPNQQVLRGYGEDNVKYIAAVAERGDPSASIPPVVTSWAQMTRETGGLTSVWESANTTSSHALMSQLSSLHVGF

(19) United States
(12) Patent Application Publication
(10) Pub. No.: US 2010/0074991 A1
(43) Pub. Date: Mar. 25, 2010

(54) NOVEL OXIDOREDUCTASES AND USES THEREOF

(76) Inventors: Johanna Henrica Gerdina Maria Mutsaers, Den Hoorn (NL); Roelf Bernhard Meima, Kamerik (NL); Albertus Alard van Dijk, Vlaardingen (NL); Natalja Alekseevna Cyplenkova, Vlaardingen (NL); Petrus Jacobus Theodorus Dekker, Den Haag (NL)

Correspondence Address: NIXON & VANDERHYE, PC, 901 NORTH GLEBE ROAD, 11TH FLOOR, ARLINGTON, VA, 22203, US

(21) Appl. No.: 12/278,362

(22) PCT Filed: Feb. 6, 2007
(86) PCT No.: PCT/EP07/01134

§ 371 Date: Jun. 3, 2009

(30) Foreign Application Priority Data
Feb. 6, 2006 (EP) 2006/050700

(51) Int. Cl. C12N 9/04; C07H 21/00; C12N 15/74; C12P 19/34; C12N 15/64; C12P 21/00; C12N 1/15; C07K 16/00; A21D 8/04; A23C 19/032; A23C 9/12

(52) U.S. Cl. 426/20; 536/23.1; 435/320.1; 435/91.1; 435/91.4; 435/190; 435/69.1; 435/254.3; 530/387.1; 426/42; 426/36

(57) ABSTRACT

The invention relates to newly identified polynucleotide sequences comprising a gene that encodes a novel oxidoreductase isolated from Aspergillus niger. The invention features the full length nucleotide sequence of the novel gene, the cDNA sequence comprising the full length coding sequence of the novel oxidoreductase as well as the amino acid sequence of the full-length functional protein and functional equivalents thereof. The invention also relates to methods of using these enzymes in baking and in dairy applications. Also included in the invention are cells transformed with a polynucleotide according to the invention and cells wherein a oxidoreductase according to the invention is genetically modified to enhance or reduce its activity and/or level of expression.

### FIELD OF THE INVENTION

The invention relates to newly identified polynucleotide sequences comprising genes that encode novel oxidoreductases isolated from Aspergillus niger. The invention features the full length nucleotide sequence of the novel genes, the cDNA sequence comprising the full length coding sequences of the novel oxidoreductases as well as the amino acid sequence of the full-length functional protein and functional equivalents thereof. The invention also relates to methods of using these enzymes in baking and dairy applications. Also included in the invention are cells transformed with a polynucleotide according to the invention and cells wherein an oxidoreductase according to the invention is genetically modified to enhance or reduce its activity and/or level of expression.

### BACKGROUND OF THE INVENTION

Oxidoreductases are defined herein as enzymes that catalyse oxido-reduction reactions. The class of enzymes known as oxidoreductases (EC 1.#.#.# whereby # is a number) is defined by the Nomenclature Committee of the International Union of Biochemistry on the Nomenclature and Classification of Enzymes (Enzyme Nomenclature, Academic Press, New York, 1992) as all enzymes which catalyze oxidoreduction reactions. The substrate oxidized is regarded as a hydrogen or electron donor. The second number in the code indicates the group in the hydrogen donor that undergoes oxidation. The third number indicates the type of acceptor involved, a 3 meaning that oxygen is the acceptor. The substrate that is oxidized is regarded as hydrogen donor.

Examples of oxidoreductases are:
Laccase (EC 1.10.3.2) catalyses the oxidation of both o- and p-quinols, and are often also acting on aminophenols and phenylenediamine.
Glucose oxidase (EC 1.1.3.4-GOX) catalyses the oxidation of glucose and several other sugars.
Hexose oxidase (EC 1.1.3.5) catalyses the same reaction as GOX, namely, the oxidation of glucose and several other sugars such as galactose, mannose, maltose, lactose and cellobiose.
Cholesterol oxidase (EC 1.1.3.6) catalyses the oxidoreduction of cholesterol.
Choline dehydrogenase (E.C. 1.1.99.1) catalyses the oxidoreduction of choline.
Glucose dehydrogenase (E.C. 1.1.99.10) catalyses the oxidoreduction of glucose.
Alcohol oxidase (EC 1.1.3.13) catalyses the oxidation of primary alcohols.
Secondary-alcohol oxidase (EC 1.1.3.18) catalyses the oxidation of secondary alcohols.
D-aspartate oxidase (EC 1.4.3.1) catalyses the oxidoreduction of aspartase and is also known as aspartic oxidase.
Putrescine oxidase (EC 1.4.3.10) catalyses the oxidoreduction of putrescine into 4-aminobutanal which condenses into 1-pyrroline.
Amine oxidase (EC 1.4.3.4) catalyses the oxidoreduction of amines, mainly primary amines and usually some secondary and tertiary amines.
Sarcosine oxidase (EC 1.5.3.1) catalyses the oxidoreduction of sarcosine.
Polyamine oxidase (EC 1.5.3.11) catalyses the oxidoreduction of N¹-acetylspermine.
(R)-6-Hydroxynicotine oxidase (EC 1.5.3.6) catalyses the oxidoreduction of (R)-6-Hydroxynicotine.
Reticuline oxidase (EC 1.5.3.9) catalyses the oxidoreduction of (S)-Reticuline which is also known as the berberine-bridge-forming enzyme.
Catechol oxidase (EC1.10.3.1) catalyses the oxidoreduction of catechol and a number of substituted catechols.
Thioredoxin reductase (EC 1.6.4.5) which catalyses the reduction of oxidised thioredoxin.
Sulfite reductase (EC 1.8.1.2) which catalyses the reduction of hydrogen sulfide.
Chloride peroxidase (EC 1.11.1.10) which brings about the chlorination of organic molecules forming stable C—Cl bonds and which can also act on Br⁻ and I⁻.
Catalase (EC 1.11.1.6) which brings about the oxidoreduction of several organic substances as for example ethanol.
Other examples of oxidoreductases are firefly luciferase (EC 1.13.12.7), cinnamic acid 4-hydroxylase (EC 1.14.13.11), benzoate 4-monooxygenase (EC 1.14.13.12), cholesterol 7α-monooxygenase (EC 1.14.13.17), pentachlorophenol monooxygenase (EC 1.14.13.50), monooxygenase (EC 1.14.14.1), steroid 11(3-monooxygenase (EC 1.14.15.4), monophenol monooxygenase (EC 1.14.18.1), prostaglandin synthase (EC 1.14.99.1) and salicylate hydroxylase (EC 1.14.13.1),
   The examples as given are in no means meant to be restrictive or limiting with respect to the present invention.

Oxidoreductases may conveniently be produced in microorganisms. Microbial oxidoreductases are available from a variety of sources; Bacillus species are a common source of bacterial enzymes, whereas fungal enzymes are commonly produced in Aspergillus species.

Microbial enzymes with oxidoreductase activity have been reported from various sources, including Penicillium, Talaromyces, Cladosporum (WO 95/29996), Trametes hirsuta (WO 97/22257). Microbial oxidoreductase genes have been cloned from several sources including Fusarium (EP 1157117 A1), Coriolus versicolor (DE 195 45 780 A1), Trametes (U.S. Pat. No. 6,146,865), Trichoderma (U.S. Pat. No. 6,248,575) and Microdochium nivale (WO9931990).

Oxidoreductases can be used in all application areas where also chemical oxidizing agents are used. Such chemical oxidizing agents are usually non-specific oxidants, such as iodates, peroxides, ascorbic acid, potassium bromate and azodicarbonamide. However, the use of several of the currently available chemical oxidizing agents has met consumer resistance or is not permitted by regulatory agencies, especially in the area of food applications.

The use of oxidoreductases has been considered as an alternative to chemical oxidizing agents.

Oxidoreductases may be used in a manifold of industrial applications, including food preparation and detergents.

The above-mentioned industrial applications of the oxidoreductase enzyme are only a few examples and this listing is not meant to be restrictive.

One example of food preparation in which oxidoreductases can be conveniently used is in the field of baking applications for example to improve dough or baked product quality.

For example U.S. Pat. No. 2,783,150 discloses the use of GOX in flour to improve dough strength, and texture and appearance of baked bread. EP0338452 discloses the application of glucose oxidase in combination with hemicellulose and/or cellulose degrading enzymes. WO02/30207 discloses the use of GOX in baking in combination with protein disulfide isomerase to improve the effectiveness of GOX. WO96/39851 discloses the use of the hexose oxidase of the red seaweed Chondrus crispus in baking applications. WO98/44804 discloses the use of a glycerol oxidase for improvement of rheological properties of dough.

Other food preparations in which oxidoreductases can be used include dairy foods. For example WO 02/39828 discloses the use of hexose oxidase in order to reduce Maillard reactions in pizza cheese during pizza preparation and WO 99/31990 discloses the use of carbohydrate oxidase to produce lactobionic acid from lactose, the most abundant sugar in milk.

Presently, glucose oxidase from Aspergillus niger is the only enzyme which is used in industry for the applications above. The problem with the other oxidoreductases is that their production still cannot be carried out in a cost-effective manner and/or that their properties are not optimal for their intended use. Therefore, there is still a drive for improvement of the oxidoreductases for specific applications, for example in view of effectiveness, substrate specificity and/or affinity, stability and activity at the required temperature ranges and pH ranges etcetera.

More specifically, for baking applications, the oxidoreductases used in baking today (predominantly glucose oxidase from Aspergillus niger) do not have the performance of for example potassium bromate. Potassium bromate is a chemical oxidizing agent, which is considered technically an outstanding oxidizer, especially for long baking processes. The banning of bromate leaves bakers with a gap in performance that is not yet filled today. Another example is enzymatic crumb bleaching in order to get a nice white bread crumb. For many years, enzyme active soy flour containing lipoxygenase has been used for this purpose. Introduction of genetically modified soy varieties into the market initiated a world-wide consumer resistance against the use of such soy flour in baking. As an alternative other bean and pea flours may be used, however, these are not as effective as soy flour. Therefore, also for the crumb bleaching application there is still a great need for an enzymatic solution of this problem.

More specifically, for dairy applications, a slight modification or deviation in process parameters during the heat treatment leads to an increased colour development, resulting from increased Maillard reactions. This colour development is undesired, and there is a need to control this browning process other than via a tight temperature and process control in order to make the pasteurisation process more robust.

Also in heat treatment of cheese, the browning of cheese, for example on a pizza is undesirable. WO 02/39828 summarizes several of attempts to reduce the browning, which usually aim to obtain either a very tight process control or process modifications of the cheese manufacturing process. The disadvantage of these solutions is that they are difficult to handle and/or may increase cost or decrease yield.

The present invention addresses at least one if not all of the above problems.

### OBJECT OF THE INVENTION

It is an object of the invention to provide novel polynucleotides encoding novel oxidoreductases with improved properties. A further object is to provide naturally and recombinantly produced oxidoreductases as well as recombinant strains producing these. Also methods of making and using the polynucleotides and polypeptides according to the invention are an object of the invention.

It is also an object of the invention to provide novel oxidoreductases which solve at least one of the above-mentioned problems or to provide novel oxidoreductases, which have one or more improved properties when used in dairy, and/or baking applications.

Improved properties of dairy products can be selected from the group of reduction of browning by Maillard reaction, improved anti-microbial properties, and improved protein crosslinking.

Improved properties of dough and/or baked products can be selected from the group of increased strength of the dough, increased elasticity of the dough, increased stability of the dough, reduced stickiness of the dough, improved proofing tolerance, improved extensibility of the dough, improved machineability of the dough, increased volume of the baked product, improved crumb structure of the baked product, improved softness of the baked product, improved flavour of the baked product, improved anti-staling of the baked product, improved colour of the baked product, improved crust of the baked product or which have a broad substrate specificity.

### DETAILED DESCRIPTION OF THE INVENTION

### Polynucleotides

The invention provides for novel polynucleotides encoding novel oxidoreductase enzymes, in particular enzymes having any of the activities as mentioned above, preferably enzymes with isoamyl alcohol oxidase activity, carbohydrate oxidase, laccase, glucose oxidase, or hexose oxidase activity.

The present invention provides 6 novel polynucleotides encoding an oxidoreductase, tentatively called OXI 01, OXI 02, OXI 03, OXI 04, OXI 05, OXI 06 (hereinafter referred to as OXI 01-OXI 06), having an amino acid sequence respectively according to SEQ ID NO: 013, SEQ ID NO: 014, SEQ ID NO: 015, SEQ ID NO: 016, SEQ ID NO: 017, SEQ ID NO: 018 (hereinafter referred to as ‘SEQ ID NO: 013-018’) or functional equivalents of any of them. The sequence of the gene encoding SEQ ID NO: 013-018 was determined by sequencing a genomic clone obtained from Aspergillus niger.

It was surprisingly found that the OXI 01-OXI 06 polypeptides according to the invention improve the dough strength, when used in a process to prepare dough.

The invention provides polynucleotide sequences comprising the gene encoding the OXI 01-OXI 06 oxidoreductases (comprising respectively SEQ ID NO: 001, SEQ ID NO: 002, SEQ ID NO: 003, SEQ ID NO: 004, SEQ ID NO: 005, SEQ ID NO: 006 hereinafter referred to as ‘SEQ ID NO: 001-006’) as well as its complete cDNA sequence (respectively SEQ ID NO: 007, SEQ ID NO: 008, SEQ ID NO: 009, SEQ ID NO: 010, SEQ ID NO: 011, SEQ ID NO: 012 hereinafter referred to as ‘SEQ ID NO: 007-012’.

Accordingly, the invention relates to an isolated polynucleotide comprising the nucleotide sequence according to SEQ ID NO: 001-006 or SEQ ID NO: 007-012 or functional equivalents of any of them.

More in particular, the invention relates to an isolated polynucleotide hybridisable under stringent conditions, preferably under highly stringent conditions, to a polynucleotide according to SEQ ID NO: 001-006 or SEQ ID NO: 007-012. Advantageously, such polynucleotides may be obtained from filamentous fungi, in particular from Aspergillus niger. More specifically, the invention relates to an isolated polynucleotide having a nucleotide sequence according to SEQ ID NO: 001-006 or SEQ ID NO: 007-012.

The invention also relates to an isolated polynucleotide encoding at least one functional domain of a polypeptide according to respectively SEQ ID NO: 013-018 or functional equivalents of any of them.

For avoidance of any doubts: OXI 01 corresponds with nucleic acid sequences SEQ ID NO: 001 and SEQ ID NO: 007 and amino acid sequence SEQ ID NO: 013 and homologue functional equivalents thereof; OXI 02 corresponds with SEQ ID NO: 002, SEQ ID NO: 008 and SEQ ID NO: 014 and homologue functional equivalents thereof, etc. and OXI 06 corresponds with SEQ ID NO: 006, SEQ ID NO: 012 and SEQ ID NO: 018 and homologue functional equivalents thereof.

As used herein, the terms “gene” and “recombinant gene” refer to nucleic acid molecules which may be isolated from chromosomal DNA, which include an open reading frame encoding a protein, e.g. an A. niger oxidoreductase. A gene may include coding sequences, non-coding sequences, introns and regulatory sequences. Moreover, a gene refers to an isolated nucleic acid molecule as defined herein.

A nucleic acid molecule of the present invention, such as a nucleic acid molecule having the nucleotide sequence of SEQ ID NO: 001-006 or SEQ ID NO: 007-012 or a functional equivalent thereof, can be isolated using standard molecular biology techniques and the sequence information provided herein. For example, using all or portion of the nucleic acid sequence of SEQ ID NO: 001-006 or SEQ ID NO: 007-012 as a hybridization probe, nucleic acid molecules according to the invention can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO: 001-006 or SEQ ID NO: 007-012 can be isolated by the polymerase chain reaction (PCR) using synthetic oligonucleotide primers designed based upon the sequence information contained in SEQ ID NO: 001-006 or SEQ ID NO: 007-012.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis.

Furthermore, oligonucleotides corresponding to or hybridisable to nucleotide sequences according to the invention can be prepared by standard synthetic techniques, e.g., using an automated DNA synthesizer.

In a preferred embodiment, an isolated nucleic acid molecule of the invention comprises the nucleotide sequence shown in SEQ ID NO: 007-012. The sequence of SEQ ID NO: 007-012 corresponds to the coding region of the A. niger OXI 01-OXI 06 cDNA. This cDNA comprises sequences encoding the A. niger OXI 01-OXI 06 polypeptide according to SEQ ID NO: 013-018.

In another preferred embodiment, an isolated nucleic acid molecule of the invention comprises a nucleic acid molecule which is a complement of the nucleotide sequence shown in SEQ ID NO: 001-006 or SEQ ID NO: 007-012 or a functional equivalent of these nucleotide sequences.

A nucleic acid molecule which is complementary to another nucleotide sequence is one which is sufficiently complementary to the other nucleotide sequence such that it can hybridize to the other nucleotide sequence thereby forming a stable duplex.

One aspect of the invention pertains to isolated nucleic acid molecules that encode a polypeptide of the invention or a functional equivalent thereof such as a biologically active fragment or domain, as well as nucleic acid molecules sufficient for use as hybridisation probes to identify nucleic acid molecules encoding a polypeptide of the invention and fragments of such nucleic acid molecules suitable for use as PCR primers for the amplification or mutation of nucleic acid molecules.

An “isolated polynucleotide” or “isolated nucleic acid” is a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promotor) sequences that are immediately contiguous to the coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide that is substantially free of cellular material, viral material, or culture medium (when produced by recombinant DNA techniques), or chemical precursors or other chemicals (when chemically synthesized). Moreover, an “isolated nucleic acid fragment” is a nucleic acid fragment that is not naturally occurring as a fragment and would not be found in the natural state.

As used herein, the terms “polynucleotide” or “nucleic acid molecule” are intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. The nucleic acid may be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

Another embodiment of the invention provides an isolated nucleic acid molecule which is antisense to an OXI 01-OXI 06 nucleic acid molecule, e.g., the coding strand of an OXI 01-OXI 06 nucleic acid molecule. Also included within the scope of the invention are the complement strands of the nucleic acid molecules described herein.

### Sequencing Errors

The sequence information as provided herein should not be so narrowly construed as to require inclusion of erroneously identified bases. The specific sequences disclosed herein can be readily used to isolate the complete gene from filamentous fungi, in particular A. niger which in turn can easily be subjected to further sequence analyses thereby identifying sequencing errors.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

### Nucleic Acid Fragments, Probes and Primers

A nucleic acid molecule according to the invention may comprise only a portion or a fragment of the nucleic acid sequence shown in SEQ ID NO: 001-006 or SEQ ID NO: 007-012, for example a fragment which can be used as a probe or primer or a fragment encoding a portion of an OXI 01-OXI 06 protein. The nucleotide sequence determined from the cloning of the OXI 01-OXI 06 gene and cDNA allows for the generation of probes and primers designed for use in identifying and/or cloning other OXI 01-OXI 06 family members, as well as OXI 01-OXI 06 homologues from other species. The probe/primer typically comprises substantially purified oligonucleotide which typically comprises a region of nucleotide sequence that hybridizes preferably under highly stringent conditions to at least about 12 or 15, preferably about 18 or 20, preferably about 22 or 25, more preferably about 30, 35, 40, 45, 50, 55, 60, 65, or 75 or more consecutive nucleotides of a nucleotide sequence shown in SEQ ID NO: 001-006 or SEQ ID NO: 007-012 or of a functional equivalent thereof.

Probes based on the OXI 01-OXI 06 nucleotide sequences can be used to detect transcripts or genomic OXI 01-OXI 06 sequences encoding the same or homologous proteins for instance in other organisms. In preferred embodiments, the probe further comprises a label group attached thereto, e.g., the label group can be a radioisotope, a fluorescent compound, an enzyme, or an enzyme cofactor. Such probes can also be used as part of a diagnostic test kit for identifying cells which express an OXI 01-OXI 06 protein.

### Identity & Homology

The terms “homology” or “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length.

The skilled person will be aware of the fact that several different computer programs are available to determine the homology between two sequences. For instance, a comparison of sequences and determination of percent identity between two sequences can be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity two amino acid or nucleotide sequence is determined using the algorithm of E. Meyers and W. Miller (CABIOS, 4:11-17 (1989) which has been incorporated into the ALIGN program (version 2.0) (available at: http://vega.igh.cnrs.fr/bin/align-guess.cgi) using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to OXI 01-OXI 06 nucleic acid molecules of the invention. BLAST protein searches can be performed with the XBLAST program, score=50, wordlength=3 to obtain amino acid sequences homologous to OXI 01-OXI 06 protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See http://www.ncbi.nlm.nih.gov.

### Hybridisation

As used herein, the term “hybridizing” is intended to describe conditions for hybridization and washing under which nucleotide sequences at least about 50%, at least about 40%, at least about 70%, more preferably at least about 80%, even more preferably at least about 85% to 90%, more preferably at least 95% homologous to each other typically remain hybridized to each other.

A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° C., preferably at 60° C. and even more preferably at 65° C.

Highly stringent conditions include, for example, hybridizing at 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridisation conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

### Obtaining Full Length DNA from Other Organisms

In a typical approach, cDNA libraries constructed from other organisms, e.g. filamentous fungi, in particular from the species Aspergillus can be screened.

For example, Aspergillus strains can be screened for homologous OXI 01-OXI 06 polynucleotides by Northern blot analysis. Upon detection of transcripts homologous to polynucleotides according to the invention, cDNA libraries can be constructed from RNA isolated from the appropriate strain, utilizing standard techniques well known to those of skill in the art. Alternatively, a total genomic DNA library can be screened using a probe hybridisable to an OXI 01-OXI 06 polynucleotide according to the invention.

Homologous gene sequences can be isolated, for example, by performing PCR using two degenerate oligonucleotide primer pools designed on the basis of nucleotide sequences as taught herein.

The template for the reaction can be cDNA obtained by reverse transcription of mRNA prepared from strains known or suspected to express a polynucleotide according to the invention. The PCR product can be subcloned and sequenced to ensure that the amplified sequences represent the sequences of a new OXI 01-OXI 06 nucleic acid sequence, or a functional equivalent thereof.

The PCR fragment can then be used to isolate a full-length cDNA clone by a variety of known methods. For example, the amplified fragment can be labelled and used to screen a bacteriophage or cosmid cDNA library. Alternatively, the labelled fragment can be used to screen a genomic library.

PCR technology also can be used to isolate full-length cDNA sequences from other organisms. For example, RNA can be isolated, following standard procedures, from an appropriate cellular or tissue source. A reverse transcription reaction can be performed on the RNA using an oligonucleotide primer specific for the most 5′ end of the amplified fragment for the priming of first strand synthesis.

The resulting RNA/DNA hybrid can then be “tailed” (e.g., with guanines) using a standard terminal transferase reaction, the hybrid can be digested with RNase H, and second strand synthesis can then be primed (e.g., with a poly-C primer). Thus, cDNA sequences upstream of the amplified fragment can easily be isolated. For a review of useful cloning strategies, see e.g., Sambrook et al., supra; and Ausubel et al., supra.

Whether or not a homologous DNA fragment encodes a functional OXI 01-OXI 06 protein, may easily be tested by methods known in the art.

### Vectors

Another aspect of the invention pertains to vectors, preferably expression vectors, containing a nucleic acid encoding an OXI 01-OXI 06 protein or a functional equivalent thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vector includes one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signal). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in a certain host cell (e.g. tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, encoded by nucleic acids as described herein (e.g. OXI 01-OXI 06 proteins, mutant forms of OXI 01-OXI 06 proteins, fragments, variants or functional equivalents of any of them, etc.).

The recombinant expression vectors of the invention can be designed for expression of OXI 01-OXI 06 proteins in prokaryotic or eukaryotic cells. For example, OXI 01-OXI 06 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression vectors useful in the present invention include chromosomal-, episomal- and virus-derived vectors e.g., vectors derived from bacterial plasmids, bacteriophage, yeast episome, yeast chromosomal elements, viruses such as baculoviruses, papova viruses, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled person. In a specific embodiment, promoters are preferred that are capable of directing a high expression level of oxidoreductases in filamentous fungi. Such promoters are known in the art. The expression constructs may contain sites for transcription initiation, termination, and, in the transcribed region, a ribosome binding site for translation. The coding portion of the mature transcripts expressed by the constructs will include a translation initiating AUG at the beginning and a termination codon appropriately positioned at the end of the polypeptide to be translated.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, transduction, infection, lipofection, cationic lipid mediated transfection or electroporation. Suitable methods for transforming or transfecting host cells can be found in Sambrook, et al. (Molecular Cloning: A Laboratory Manual, 2^{nd}, ed. Cold Spring Harbor Laboratory, Cold Spring Harbor Laboratory Press, Cold Spring Harbor, N.Y., 1989), Davis et al., Basic Methods in Molecular Biology (1986) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methatrexate. Nucleic acid encoding a selectable marker can be introduced into a host cell on the same vector as that encoding an OXI 01-OXI 06 protein or can be introduced on a separate vector. Cells stably transfected with the introduced nucleic acid can be identified by drug selection (e.g. cells that have incorporated the selectable marker gene will survive, while the other cells die).

Expression of proteins in prokaryotes is often carried out in E. coli with vectors containing constitutive or inducible promoters directing the expression of proteins.

As indicated, the expression vectors will preferably contain selectable markers. Such markers include dihydrofolate reductase or neomycin resistance for eukarotic cell culture and tetracyline or ampicilling resistance for culturing in E. coli and other bacteria. Representative examples of appropriate host include bacterial cells, such as E. coli, Streptomyces and Salmonella typhimurium; fungal cells, such as yeast; insect cells such as Drosophila S2 and Spodoptera Sf9; animal cells such as CHO, COS and Bowes melanoma; and plant cells. Appropriate culture media and conditions for the above-described host cells are known in the art.

Among vectors preferred for use in bacteria are pQE70, pQE60 and PQE-9, available from Qiagen; pBS vectors, Phagescript vectors, Bluescript vectors, pNH8A, pNH16A, pNH18A, pNH46A, available from Stratagene; and ptrc99a, pKK223-3, pKK233-3, pDR540, pRIT5 available from Pharmacia. Among preferred eukaryotic vectors are PWLNEO, pSV2CAT, pOG44, pZT1 and pSG available from Stratagene; and pSVK3, pBPV, pMSG and pSVL available from Pharmacia. Other suitable vectors will be readily apparent to the skilled artisan.

Among known bacterial promotors for use in the present invention include E. coli lacI and lacZ promoters, the T3 and T7 promoters, the gpt promoter, the lambda PR, PL promoters and the trp promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

Transcription of the DNA encoding the polypeptides of the present invention by higher eukaryotes may be increased by inserting an enhancer sequence into the vector. Enhancers are cis-acting elements of DNA, usually about from 10 to 300 by that act to increase transcriptional activity of a promoter in a given host cell-type. Examples of enhancers include the SV40 enhancer, which is located on the late side of the replication origin at by 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretation signal may be incorporated into the expressed polypeptide. The signals may be endogenous to the polypeptide or they may be heterologous signals.

The polypeptide may be expressed in a modified form and may include not only secretion signals but also additional heterologous functional regions. Thus, for instance, a region of additional amino acids, particularly charged amino acids, may be added to the N-terminus of the polypeptide to improve stability and persistence in the host cell, during purification or during subsequent handling and storage. Also, peptide moieties may be added to the polypeptide to facilitate purification.

### Polypeptides According to the Invention

The invention provides an isolated polypeptide having the amino acid sequence according to SEQ ID NO: 013-018, an amino acid sequence obtainable by expressing the polynucleotide of SEQ ID NO: 001-077 in an appropriate host, as well as an amino acid sequence obtainable by expressing the polynucleotide sequences of SEQ ID NO: 007-012 in an appropriate host. Also, a peptide or polypeptide comprising a functional equivalent of the above polypeptides is comprised within the present invention. The above polypeptides are collectively comprised in the term “polypeptides according to the invention”

The terms “peptide” and “oligopeptide” are considered synonymous (as is commonly recognized) and each term can be used interchangeably as the context requires to indicate a chain of at least two amino acids coupled by peptidyl linkages. The word “polypeptide” is used herein for chains containing more than seven amino acid residues. All oligopeptide and polypeptide formulas or sequences herein are written from left to right and in the direction from amino terminus to carboxy terminus. The one-letter code of amino acids used herein is commonly known in the art and can be found in Sambrook, et al. (Molecular Cloning: A Laboratory Manual, 2^{nd},ed. Cold Spring Harbor Laboratory, Cold Spring Harbor Laboratory Press, Cold Spring Harbor, N.Y., 1989)

By “isolated” polypeptide or protein is intended a polypeptide or protein removed from its native environment. For example, recombinantly produced polypeptides and proteins expressed in host cells are considered isolated for the purpose of the invention as are native or recombinant polypeptides which have been substantially purified by any suitable technique such as, for example, the single-step purification method disclosed in Smith and Johnson, Gene 67:31-40 (1988).

The OXI 01-OXI 06 oxidoreductase according to the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

### Protein Fragments

The invention also features biologically active fragments of the polypeptides according to the invention.

Biologically active fragments of a polypeptide of the invention include polypeptides comprising amino acid sequences sufficiently identical to or derived from the amino acid sequence of the OXI 01-OXI 06 protein (e.g., the amino acid sequence of SEQ ID NO: 013-018), which include fewer amino acids than the full length protein, and exhibit at least one biological activity of the corresponding full-length protein. Typically, biologically active fragments comprise a domain or motif with at least one activity of the OXI 01-OXI 06 protein. Preferred is a fragment with glucose oxidase activity (E.C. 1.1.3.4).

A biologically active fragment of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the biological activities of the native form of a polypeptide of the invention.

The invention also features nucleic acid fragments which encode the above biologically active fragments of the OXI 01-OXI 06 protein.

### Functional Equivalents

The terms “functional equivalents” and “functional variants” are used interchangeably herein. Functional equivalents of OXI 01-OXI 06 DNA are isolated DNA fragments that encode a polypeptide that exhibits a particular function of the OXI 01-OXI 06 A. niger oxidoreductase as defined herein. A functional equivalent of an OXI 01-OXI 06 polypeptide according to the invention is a polypeptide that exhibits at least one function of an A. niger oxidoreductase as defined herein. Functional equivalents therefore also encompass biologically active fragments.

Functional protein or polypeptide equivalents may contain only conservative substitutions of one or more amino acids of SEQ ID NO: 013-018 or substitutions, insertions or deletions of non-essential amino acids. Accordingly, a non-essential amino acid is a residue that can be altered in SEQ ID NO: 013-018 without substantially altering the biological function. For example, amino acid residues that are conserved among the OXI 01-OXI 06 proteins of the present invention, are predicted to be particularly unamenable to alteration. Furthermore, amino acids conserved among the OXI 01-OXI 06 proteins according to the present invention and other oxidoreductases are not likely to be amenable to alteration.

The term “conservative substitution” is intended to mean that a substitution in which the amino acid residue is replaced with an amino acid residue having a similar side chain. These families are known in the art and include amino acids with basic side chains (e.g., lysine, arginine and hystidine), acidic side chains (e.g. aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine tryptophan, histidine).

Functional nucleic acid equivalents may typically contain silent mutations or mutations that do not alter the biological function of encoded polypeptide. Accordingly, the invention provides nucleic acid molecules encoding OXI 01-OXI 06 proteins that contain changes in amino acid residues that are not essential for a particular biological activity. Such OXI 01-OXI 06 proteins differ in amino acid sequence from SEQ ID NO: 013-018 yet retain at least one biological activity. Accordingly, the invention also encompasses isolated nucleic acid molecules comprising a nucleotide sequence encoding a protein, wherein the protein comprises a substantially homologous amino acid sequence of at least about 63%, 65%, 70%, 75%, 80%, 85%, 90%, 95%, 96%, 97%, 98%, 99% or more homologous to the amino acid sequence shown in SEQ ID NO: 013 In another embodiment the isolated nucleic acid molecule comprises a nucleotide sequence encoding a protein, wherein the protein comprises a substantially homologous amino acid sequence of at least about 40%, 65%, 70%, 75%, 80%, 85%, 90%, 95%, 96%, 97%, 98%, 99% or more homologous to the amino acid sequence shown in SEQ ID NO: 014-018.

For example, guidance concerning how to make phenotypically silent amino acid substitutions is provided in Bowie, J. U. et al., Science 247:1306-1310 (1990) wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As the authors state, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, supra, and the references cited therein.

An isolated nucleic acid molecule encoding an OXI 01-OXI 06 protein homologous to the protein according to SEQ ID NO: 013-018 can be created by introducing one or more nucleotide substitutions, additions or deletions into the coding nucleotide sequences according to SEQ ID NO: 001-006 or SEQ ID NO: 007-012 such that one or more amino acid substitutions, deletions or insertions are introduced into the encoded protein. Such mutations may be introduced by standard techniques, such as site-directed mutagenesis and PCR-mediated mutagenesis.

The term “functional equivalents” also encompasses orthologues of the A. niger OXI 01-OXI 06 protein. Orthologues of the A. niger OXI 01-OXI 06 protein are proteins that can be isolated from other strains or species and possess a similar or identical biological activity. Such orthologues can readily be identified as comprising an amino acid sequence that is substantially homologous to SEQ ID NO: 013-018.

As defined herein, the term “substantially homologous” refers to a first amino acid or nucleotide sequence which contains a sufficient or minimum number of identical or equivalent (e.g., with similar side chain) amino acids or nucleotides to a second amino acid or nucleotide sequence such that the first and the second amino acid or nucleotide sequences have a common domain. For example, amino acid or nucleotide sequences which contain a common domain having about 40%, preferably 65%, more preferably 70%, even more preferably 75%, 80%, 85%, 90%, 95%, 96%, 97%, 98% or 99% identity or more are defined herein as sufficiently identical.

Also, nucleic acids encoding other OXI 01-OXI 06 family members, which thus have a nucleotide sequence that differs from SEQ ID NO: 001-006 or SEQ ID NO: 007-012, are within the scope of the invention. Moreover, nucleic acids encoding OXI 01-OXI 06 proteins from different species which thus have a nucleotide sequence which differs from SEQ ID NO: 001-006 or SEQ ID NO: 007-012 are within the scope of the invention.

Nucleic acid molecules corresponding to variants (e.g. natural allelic variants) and homologues of the OXI 01-OXI 06 DNA of the invention can be isolated based on their homology to the OXI 01-OXI 06 nucleic acids disclosed herein using the cDNAs disclosed herein or a suitable fragment thereof, as a hybridisation probe according to standard hybridisation techniques preferably under highly stringent hybridisation conditions.

In addition to naturally occurring allelic variants of the OXI 01-OXI 06 sequence, the skilled person will recognise that changes can be introduced by mutation into the nucleotide sequences of SEQ ID NO: 001-006 or SEQ ID NO: 007-012 thereby leading to changes in the amino acid sequence of the OXI 01-OXI 06 protein without substantially altering the function of the OXI 01-OXI 06 protein.

In another aspect of the invention, improved OXI 01-OXI 06 proteins are provided. Improved OXI 01-OXI 06 proteins are proteins wherein at least one biological activity is improved. Such proteins may be obtained by randomly introducing mutations along all or part of the OXI 01-OXI 06 coding sequence, such as by saturation mutagenesis, and the resulting mutants can be expressed recombinantly and screened for biological activity. For instance, the art provides for standard assays for measuring the enzymatic activity of oxidoreductases and thus improved proteins may easily be selected.

In a preferred embodiment the OXI 01-OXI 06 protein has an amino acid sequence according to SEQ ID NO: 013-018. In another embodiment, the OXI 01-OXI 06 polypeptide is substantially homologous to the amino acid sequence according to SEQ ID NO: 013-018 and retains at least one biological activity of a polypeptide according to SEQ ID NO: 013-018, yet differs in amino acid sequence due to natural variation or mutagenesis as described above.

In a further preferred embodiment, the OXI 01-OXI 06 protein has an amino acid sequence encoded by an isolated nucleic acid fragment capable of hybridising to a nucleic acid according to SEQ ID NO: 001-006 or SEQ ID NO: 007-012, preferably under highly stringent hybridisation conditions.

For the protein comprising the amino acid sequence according to SEQ ID NO: 013, the closest homolog to a functional enzyme is isoamyl alcohol oxidase from Aspergillus fumigatus, which shows 62% identity to SEQ ID 013. In one embodiment the isolated nucleic acid molecule comprises a nucleotide sequence encoding a protein, wherein the protein comprises a substantially homologous amino acid sequence of at least about 63%, 65%, 70%, 75%, 80%, 85%, 90%, 95%, 96%, 97%, 98%, 99% or more homologous to the amino acid sequence shown in SEQ ID NO: 013, and being a functional equivalent of the protein comprising the amino acid sequence according to SEQ ID NO: 013.

For the protein comprising the amino acid sequence according to SEQ ID NO: 014, the closest homolog to a functional enzyme is 6-hydroxy-D-nicotine oxidase from Anthrobacter oxidans, which shows xx % identities to SEQ ID 014. In one embodiment the isolated nucleic acid molecule comprises a nucleotide sequence encoding a protein, wherein the protein comprises a substantially homologous amino acid sequence of at least about 40%, 65%, 70%, 75%, 80%, 85%, 90%, 95%, 96%, 97%, 98%, 99% or more homologous to the amino acid sequence shown in SEQ ID NO: 014, and being a functional equivalent of the protein comprising the amino acid sequence according to SEQ ID NO: 014.

For the protein comprising the amino acid sequence according to SEQ ID NO: 015, the closest homolog to a functional enzyme is versicolorin B synthase form Aspergillus parasiticus which shows 31% identities to SEQ ID 015. In one embodiment the isolated nucleic acid molecule comprises a nucleotide sequence encoding a protein, wherein the protein comprises a substantially homologous amino acid sequence of at least about 40%, 65%, 70%, 75%, 80%, 85%, 90%, 95%, 96%, 97%, 98%, 99% or more homologous to the amino acid sequence shown in SEQ ID NO: 015, and being a functional equivalent of the protein comprising the amino acid sequence according to SEQ ID NO: 015.

The protein comprising the amino acid sequence according to SEQ ID NO: 016 shows no homology to any functional enzyme. In one embodiment the isolated nucleic acid molecule comprises a nucleotide sequence encoding a protein, wherein the protein comprises a substantially homologous amino acid sequence of at least about 40%, 65%, 70%, 75%, 80%, 85%, 90%, 95%, 96%, 97%, 98%, 99% or more homologous to the amino acid sequence shown in SEQ ID NO: 016, and being a functional equivalent of the protein comprising the amino acid sequence according to SEQ ID NO: 016. For the protein comprising the amino acid sequence according to SEQ ID NO: 017 the closest homolog to a functional enzyme is 6-hydroxy-D-nicotine oxidase from Arthrobacter oxidans which shows <25% identity to SEQ ID 017. In one embodiment the isolated nucleic acid molecule comprises a nucleotide sequence encoding a protein, wherein the protein comprises a substantially homologous amino acid sequence of at least about 40%, 65%, 70%, 75%, 80%, 85%, 90%, 95%, 96%, 97%, 98%, 99% or more homologous to the amino acid sequence shown in SEQ ID NO: 017, and being a functional equivalent of the protein comprising the amino acid sequence according to SEQ ID NO: 017.

The protein comprising the amino acid sequence according to SEQ ID NO: 018 shows no homology to a functional enzyme. In one embodiment the isolated nucleic acid molecule according to the invention comprises a nucleotide sequence encoding a protein, wherein the protein comprises a substantially homologous amino acid sequence of at least about 40%, 65%, 70%, 75%, 80%, 85%, 90%, 95%, 96%, 97%, 98%, 99% or more homologous to the amino acid sequence shown in SEQ ID NO: 018, and being a functional equivalent of the protein comprising the amino acid sequence according to SEQ ID NO: 018.

Accordingly, the OXI 01 protein is a protein which comprises an amino acid sequence at least about 63%, 65%, 70%, 75%, 80%, 85%, 90%, 95%, 96%, 97%, 98%, 99% or more homologous to the amino acid sequence shown in SEQ ID NO: 013 and retains at least one functional activity of the polypeptide according to SEQ ID NO: 013

Analogous, the OXI2-OXI 06 proteins are proteins which comprise an amino acid sequence at least about 40%, 65%, 70%, 75%, 80%, 85%, 90%, 95%, 96%, 97%, 98%, 99% or more homologous to the amino acid sequence shown in SEQ ID NO: 014-018 respectively, and retain at least one functional activity of the polypeptide according to SEQ ID NO: 014-018.

Functional equivalents of a protein according to the invention can also be identified e.g. by screening combinatorial libraries of mutants, e.g. truncation mutants, of the protein of the invention for oxidoreductase activity. In one embodiment, a variegated library of variants is generated by combinatorial mutagenesis at the nucleic acid level. A variegated library of variants can be produced by, for example, enzymatically ligating a mixture of synthetic oligonucleotides into gene sequences such that a degenerate set of potential protein sequences is expressible as individual polypeptides. There are a variety of methods that can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide of the invention can be used to generate a variegated population of polypeptides for screening a subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations of truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

In addition to the OXI 01-OXI 06 gene sequence shown in SEQ ID NO: 1, it will be apparent for the person skilled in the art that DNA sequence polymorphisms that may lead to changes in the amino acid sequence of the OXI 01-OXI 06 protein may exist within a given population. Such genetic polymorphisms may exist in cells from different populations or within a population due to natural allelic variation. Allelic variants may also include functional equivalents.

Fragments of a polynucleotide according to the invention may also comprise polynucleotides not encoding functional polypeptides. Such polynucleotides may function as probes or primers for a PCR reaction.

Nucleic acids according to the invention irrespective of whether they encode functional or non-functional polypeptides, can be used as hybridization probes or polymerase chain reaction (PCR) primers. Uses of the nucleic acid molecules of the present invention that do not encode a polypeptide having an OXI 01-OXI 06 activity include, inter alia, (1) isolating the gene encoding the OXI 01-OXI 06 protein, or allelic variants thereof from a cDNA library e.g. from other organisms than A. niger; (2) in situ hybridization (e.g. FISH) to metaphase chromosomal spreads to provide precise chromosomal location of the OXI 01-OXI 06 gene as described in Verma et al., Human Chromosomes: a Manual of Basic Techniques, Pergamon Press, New York (1988); (3) Northern blot analysis for detecting expression of OXI 01-OXI 06 mRNA in specific tissues and/or cells and 4) probes and primers that can be used as a diagnostic tool to analyse the presence of a nucleic acid hybridisable to the OXI 01-OXI 06 probe in a given biological (e.g. tissue) sample.

Also encompassed by the invention is a method of obtaining a functional equivalent of an OXI 01-OXI 06 gene or cDNA. Such a method entails obtaining a labelled probe that includes an isolated nucleic acid which encodes all or a portion of the sequence according to SEQ ID NO: 013-018 or a variant thereof; screening a nucleic acid fragment library with the labelled probe under conditions that allow hybridisation of the probe to nucleic acid fragments in the library, thereby forming nucleic acid duplexes, and preparing a full-length gene sequence from the nucleic acid fragments in any labelled duplex to obtain a gene related to the OXI 01-OXI 06 gene.

### Host Cells

In another embodiment, the invention features cells, e.g., transformed host cells or recombinant host cells that contain a nucleic acid encompassed by the invention. A “transformed cell” or “recombinant cell” is a cell into which (or into an ancestor of which) has been introduced, by means of recombinant DNA techniques, a nucleic acid according to the invention. Both prokaryotic and eukaryotic cells are included, e.g., bacteria, fungi, yeast, and the like, especially preferred are cells from filamentous fungi, in particular Aspergillus niger.

A host cell can be chosen that modulates the expression of the inserted sequences, or modifies and processes the gene product in a specific, desired fashion. Such modifications (e.g., glycosylation) and processing (e.g., cleavage) of protein products may facilitate optimal functioning of the protein.

Various host cells have characteristic and specific mechanisms for post-translational processing and modification of proteins and gene products. Appropriate cell lines or host systems familiar to those of skill in the art of molecular biology and/or microbiology can be chosen to ensure the desired and correct modification and processing of the foreign protein expressed. To this end, eukaryotic host cells that possess the cellular machinery for proper processing of the primary transcript, glycosylation, and phosphorylation of the gene product can be used. Such host cells are well known in the art.

Host cells also include, but are not limited to, mammalian cell lines such as CHO, VERO, BHK, HeLa, COS, MDCK, 293, 3T3, WI38, and choroid plexus cell lines.

If desired, the polypeptides according to the invention can be produced by a stably-transfected cell line. A number of vectors suitable for stable transfection of mammalian cells are available to the public, methods for constructing such cell lines are also publicly known, e.g., in Ausubel et al. (supra).

### Use of Oxidoreductases in Industrial Processes

The invention also relates to the use of the oxidoreductase according to the invention in a selected number of industrial processes. Despite the long-term experience obtained with these processes, the oxidoreductase according to the invention features a number of significant advantages over the enzymes currently used. Depending on the specific application, these advantages can include aspects like better performance, lower production costs, higher specificity towards the substrate, being less antigenic, less undesirable side activities, higher yields when produced in a suitable microorganism, more suitable pH and temperature ranges, better tastes of the final product as well as food grade and kosher aspects.

The oxidoreductase of the present invention may be used in any application where it is desired to oxidise a substrate or to obtain specific reaction products thereof. For example application of the oxidoreductase according to the invention can yield hydrogen peroxide together with aldehydes, alcohols, carboxylic acid etcetera.

One of the industrial processes the novel oxidoreductase according the invention can be used for is for baking applications. The invention also relates to a method of providing flour doughs having improved rheological properties and to finished baked or dried products made from such doughs, which have improved textural, eating quality and dimensional characteristics. The invention also relates to a baking premix, which comprises flour, an enzyme preparation and a suitable carrier.

The invention results in stronger doughs, with improved rheological properties as well as a final baked product with improved qualities.

The strength of dough is an important aspect of baking for both small-scale and large-scale applications. Strong dough has a greater tolerance of mixing time, proofing time, and mechanical vibrations during dough transport, whereas weak dough is less tolerant to these treatments. A strong dough with superior rheological and handling properties results from flour containing a strong gluten network. Flour with a low protein content or a poor gluten quality results in weak dough.

Dough conditioners are well known in the baking industry. The addition of conditioners to bread dough has resulted in improved machine-ability of the dough and improved texture, volume, flavor, and freshness (anti-staling) of the bread. Nonspecific oxidants, such as iodates, peroxides, ascorbic acid, potassium bromate and azodicarbonamide are used for improving the baking performance of flour, to achieve dough with improved rheological properties, and to obtain dough with a desirable strength and stability.

It has been suggested that these conditioners induce the formation of interprotein bonds which strengthen the gluten, and thereby the dough. However, the use of several of the currently available chemical oxidizing agents has met consumer resistance or is not permitted by regulatory agencies.

The use of enzymes as dough conditioners has been considered as an alternative to chemical conditioners. A number of enzymes have been used recently as dough and/or bread improving agents, in particular, enzymes that act on components present in large amounts in the dough. Examples of such enzymes are amylases, proteases, glucose oxidases, hexose oxidases, xylanases and (hemi) cellulases, including pentosanases, and lipases, phospholipases and galactolipases.

Baked products are prepared from a dough which is usually made from the basic ingredients flour, water and optionally salt. Depending on the baked products, other optional ingredients are sugars, flavours etcetera. For leavened products, primarily baker's yeast is used next to chemical leavening systems such as a combination of an acid (generating compound) and bicarbonate. In order to improve the handling properties of the dough and/or the final properties of the baked products there is a continuous effort to develop processing aids with improving properties. Dough properties that are to be improved comprise machineability, gas retaining capacity etcetera. Properties of the baked products that may be improved comprise loaf volume, crust crispiness, crumb texture and softness, taste and flavour and shelf life. The currently existing processing aids can be divided into two groups: chemical additives and enzymes.

Chemical additives with improving properties comprise chemical oxidising agents such as ascorbic acid, bromate and azodicarbonate, reducing agents such as L-cysteine and glutathione, emulsifiers acting as dough conditioners such as diacetyl tartaric esters of mono/diglycerides (DATEM), sodium stearoyl lactylate (SSL) or calcium stearoyl lactylate (CSL), or acting as crumb softeners such as glycerol monostearate (GMS) etceteras, fatty materials such as triglycerides (fat) or lecithin and others.

Presently, there is a trend to replace the chemical additives by enzymes. The latter are considered to be more natural compounds and therefore more accepted by the consumer. Suitable enzymes may be selected from oxidizing enzymes, the group consisting of starch degrading enzymes, arabinoxylan- and other hemicellulose degrading enzymes, cellulose degrading enzymes, fatty material splitting enzymes and protein degrading enzymes.

The present invention also relates to methods for preparing a dough or a baked product comprising incorporating into the dough an effective amount of a oxidoreductase of the present invention which improves one or more properties of the dough or the baked product obtained from the dough relative to a dough or a baked product in which the polypeptide is not incorporated.

The phrase “incorporating into the dough” is defined herein as adding the oxidoreductase according to the invention to the dough, any ingredient from which the dough is to be made, and/or any mixture of dough ingredients form which the dough is to be made. In other words, the oxidoreductase according to the invention may be added in any step of the dough preparation and may be added in one, two or more steps. The oxidoreductase according to the invention is added to the ingredients of dough that is kneaded and baked to make the baked product using methods well known in the art. See, for example, U.S. Pat. No. 4,567,046, EP-A-426,211, JP-A-60-78529, JP-A-62-111629, and JP-A-63-258528.

The term “effective amount” is defined herein as an amount of the oxidoreductase according to the invention that is sufficient for providing a measurable effect on at least one property of interest of the dough and/or baked product.

The term “improved property” is defined herein as any property of a dough and/or a product obtained from the dough, particularly a baked product, which is improved by the action of the oxidoreductase according to the invention relative to a dough or product in which the oxidoreductase according to the invention is not incorporated. The improved property may include, but is not limited to, increased strength of the dough, increased elasticity of the dough, increased stability of the dough, reduced stickiness of the dough, improved extensibility of the dough, improved flavour of the baked product, improved anti-staling of the baked product and improved whiteness of the crumb.

The improved property may be determined by comparison of a dough and/or a baked product prepared with and without addition of a polypeptide of the present invention in accordance with the methods of present invention are described below in the Examples. Organoleptic qualities may be evaluated using procedures well established in the baking industry, and may include, for example, the use of a panel of trained taste-testers.

The term “increased strength of the dough” is defined herein as the property of a dough that has generally more elastic properties and/or requires more work input to mould and shape.

The term “increased elasticity of the dough” is defined herein as the property of a dough which has a higher tendency to regain its original shape after being subjected to a certain physical strain.

The term “increased stability of the dough” is defined herein as the property of a dough that is less susceptible to mechanical abuse thus better maintaining its shape and volume.

The term “reduced stickiness of the dough” is defined herein as the property of a dough that has less tendency to adhere to surfaces, e.g., in the dough production machinery, and is either evaluated empirically by the skilled test baker or measured by the use of a texture analyser (e.g., TAXT2) as known in the art.

The term “improved extensibility of the dough” is defined herein as the property of a dough that can be subjected to increased strain or stretching without rupture.

The term “improved machineability of the dough” is defined herein as the property of a dough that is generally less sticky and/or more firm and/or more elastic.

The term “increased proofing resistance of a dough” is defined as the ability of the dough to withstand prolonged proofing times.

The term “increased volume of the baked product” is measured as the specific volume of a given loaf of bread (volume/weight) determined typically by the traditional rapeseed displacement method.

The term “improved crumb structure of the baked product” is defined herein as the property of a baked product with finer and/or thinner cell walls in the crumb and/or more uniform/homogenous distribution of cells in the crumb and is usually evaluated empirically by the skilled test baker.

The term “improved softness of the baked product” is the opposite of “firmness” and is defined herein as the property of a baked product that is more easily compressed and is evaluated either empirically by the skilled test baker or measured by the use of a texture analyzer (e.g., TAXT2) as known in the art.

The term “improved flavor of the baked product” is evaluated by a trained test panel.

The term “improved anti-staling of the baked product” is defined herein as the properties of a baked product that have a reduced rate of deterioration of quality parameters, e.g., softness and/or elasticity, during storage.

The term “dough” is defined herein as a mixture of flour and other ingredients firm enough to knead or roll. The dough may be fresh, frozen, pre-bared, or pre-baked. The preparation of frozen dough is described by Kulp and Lorenz in Frozen and Refrigerated Doughs and Batters.

The term “baked product” is defined herein as any product prepared from a dough, either of a soft or a crisp character. Examples of baked products, whether of a white, light or dark type, which may be advantageously produced by the present invention are bread (in particular white, whole-meal or rye bread), typically in the form of loaves or rolls, French baguette-type bread, pasta, pita bread, tortillas, tacos, cakes, pancakes, biscuits, cookies, pie crusts, steamed bread, and crisp bread, and the like.

Oxidoreductases of the present invention and/or additional enzymes to be used in the methods of the present invention may be in any form suitable for the use in question, e.g., in the form of a dry powder, agglomerated powder, or granulate, in particular a non-dusting granulate, liquid, in particular a stabilized liquid, or protected enzyme such described in WO01/11974 and WO02/26044. Granulates and agglomerated powders may be prepared by conventional methods, e.g., by spraying the oxidoreductase according to the invention onto a carrier in a fluid-bed granulator. The carrier may consist of particulate cores having a suitable particle size. The carrier may be soluble or insoluble, e.g., a salt (such as NaCl or sodium sulphate), sugar (such as sucrose or lactose), sugar alcohol (such as sorbitol), starch, rice, corn grits, or soy. The oxidoreductase according to the invention and/or additional enzymes may be contained in slow-release formulations. Methods for preparing slow-release formulations are well known in the art. Adding nutritionally acceptable stabilizers such as sugar, sugar alcohol, or another polyol, and/or lactic acid or another organic acid according to established methods may for instance, stabilize liquid enzyme preparations.

The oxidoreductases according to the invention may also be incorporated in yeast comprising compositions such as disclosed in EP-A-0619947, EP-A-0659344 and WO02/49441.

For inclusion in pre-mixes of flour it is advantageous that the polypeptide according to the invention is in the form of a dry product, e.g., a non-dusting granulate, whereas for inclusion together with a liquid it is advantageously in a liquid form.

One or more additional enzymes may also be incorporated into the dough. The additional enzyme may be of any origin, including mammalian and plant, and preferably of microbial (bacterial, yeast or fungal) origin and may be obtained by techniques conventionally used in the art.

In a preferred embodiment, the additional enzyme may be an amylase, such as an alpha-amylase (useful for providing sugars fermentable by yeast and retarding staling) or beta-amylase, cyclodextrin glucanotransferase, peptidase, in particular, an exopeptidase (useful in flavour enhancement), transglutaminase, lipase/phospholipase/galactolipase (useful for the modification of lipolytic compounds present in the dough or dough constituents), oxidoreductase, cellulase, hemicellulase, in particular a pentosanase such as xylanase (useful for the partial hydrolysis of pentosans which increases the extensibility of the dough), protease (useful for gluten weakening in particular when using hard wheat flour), protein disulfide isomerase, e.g., a protein disulfide isomerase as disclosed in WO 95/00636, glycosyltransferase, peroxidase (useful for improving the dough consistency), laccase, catechol oxidase or other oxidases, e.g., an glucose oxidase, hexose oxidase, aldose oxidase, pyranose oxidase, lipoxygenase or L-amino acid oxidase (useful in improving dough consistency).

When one or more additional enzyme activities are to be added in accordance with the methods of the present invention, these activities may be added separately or together with the polypeptide according to the invention, optionally as constituent(s) of the bread-improving and/or dough-improving composition. The other enzyme activities may be any of the enzymes described above and may be dosed in accordance with established baking practices.

The present invention also relates to methods for preparing a baked product comprising baking a dough obtained by a method of the present invention to produce a baked product. The baking of the dough to produce a baked product may be performed using methods well known in the art.

The present invention also relates to doughs and baked products, respectively, produced by the methods of the present invention.

The present invention further relates to a pre-mix, e.g., in the form of a flour composition, for dough and/or baked products made from dough, in which the pre-mix comprises a polypeptide of the present invention. The term “pre-mix” is defined herein to be understood in its conventional meaning, i.e., as a mix of baking agents, generally including flour, which may be used not only in industrial bread-baking agents, generally including flour, which may be used not only in industrial bread-baking plants/facilities, but also in retail bakeries. The pre-mix may be prepared by mixing the polypeptide or a bread-improving and/or dough-improving composition of the invention comprising the polypeptide with a suitable carrier such as flour, starch, a sugar, or a salt. The pre-mix may contain other dough-improving and/or bread-improving additives, e.g., any of the additives, including enzymes, mentioned above.

The present invention further relates to baking additives in the form of a granulate or agglomerated powder, which comprise a polypeptide of the present invention. The baking additive preferably has a narrow particle size distribution with more than 95% (by weight) of the particles in the range from 25 to 500 μm.

In dough and bread making the present invention may be used in combination with the processing aids defined hereinbefore such as the chemical processing aids like oxidants (e.g. ascorbic acid), reducing agents (e.g. L-cysteine), phospholipases and/or other enzymes such as polysaccharide modifying enzymes (e.g. α-amylase, hemicellulase, branching enzymes, etc.) and/or protein modifying enzymes (endoprotease, exoprotease, branching enzymes, etc.).

It was also found that the OXI 01 protein can produce hydrogen peroxide in dough. It surprisingly uses at least 9,12,13-trihydroxy-10-octadecenoic acid as substrate thereby producing a keto-dihydroxy-10-octadecenoic acid. This substrate is present in for example flour and makes therefore the OXI 01 protein especially suitable for baking.

The use of such an enzyme in baking was not known before. Therefore the present invention also includes the use of an enzyme that catalyzes the oxidation of a hydroxy fatty acid, e.g. a mono hydroxy fatty acid or a dihydroxy fatty acid or a trihydroxy fatty acid such as 9,12,13-trihydroxy-10-octadecenoic acid, thereby producing hydrogen peroxide, in baking. An example of a suitable type of enzyme is an alcohol oxidase, for example a secondary-alcohol oxidase or an isoamyl alcohol oxidase.

In a preferred embodiment the enzyme capable of oxidising a hydroxy fatty acid is combined with a lipoxygenase and/or a peroxidase.

The present invention also relates to a composition suitable for use in baking, comprising an enzyme capable of oxidising a hydroxy fatty acid, and a lipoxygenase and/or a peroxidase. Preferably the enzyme capable of oxidising a hydroxy fatty acid is capable of oxidising 9,12,13-trihydroxy-10-octadecenoic acid. More preferably the enzyme capable of oxidising a hydroxy fatty acid is an OXI 01 protein, even more preferably a protein comprising the amino acid sequence according to SEQ ID NO: 013.

Another use of oxidoreductases according to the present invention lies in the field of dairy applications.

Heat treatment of milk is always accompanied by some extend of Maillard reactions leading to the development of a slightly brownish color of the treated milk. Although in some cases it may be desirable (e.g. in butterscotch confections or caramel), browning is usually not desired. The Maillard reaction is the result of the reaction of reducing sugars present in milk, especially lactose, glucose and lactose, with free amino groups that are present in the milk proteins such as the caseins or the whey proteins.

The oxidoreductase OXI 01-06 according to the invention can be used to decrease Maillard reaction in milk, mild derived products or foodstuffs containing such mild derived (dairy) products at increased temperatures.

An example of such treatment is the pasteurization milk to increase its shelf life stability. In the low temperature long-time (LTLP) pasteurization, also termed vat pasteurization, the milk is typically held at 62.8° C. for not less than 30 minutes. In continuous processes, the high temperature short-time (HTST) pasteurization is used in which the milk is held at not less than 71.7° C. for a minimum of 15 seconds (or equivalent conditions at a higher temperature for a shorter time period). More recently developed is the Ultra-Heat-Treated (UHT) pasteurization in which the milk is heated to at least 135° C. for a minimum of 1 second. Especially the UHT treatment but to a lesser extend also the LTLP and HTST treatment require a very strict temperature and process control.

Another example of a suitable application to use the oxidoreductase according to the invention is the cheese spread on top of a pizza. In many cases, Mozzarella type cheese is used in pizza toppings. In the art, pasta fileta is referred to as mozzarella. Many pizza manufacturers bake pizza at temperatures>260° C. At these high temperatures the propensity of the cheese to brown excessively has become a particular concern to the mozzarella industry because the mozzarella manufacturers must deliver cheese that will not make black blisters and brown areas when baked at these high temperatures. The browning effect is typically produced by residual amounts of lactose and especially galactose. It is known in the art that there is a strong correlation coefficient between galactose and color levels of baked cheese and many attempts to reduce the level of galactose and lactose in mozzarella are mentioned in the literature. These are mostly difficult to handle and/or may increase cost or decrease yield.

Use of the oxidoreductase according to the present invention prior to the heat treatment decreases the Maillard reactions, providing an efficient route to control browning of milk during treatment at elevated temperatures. The oxidoreductase according to the present invention is preferably added in an early stage of milk treatment in order to allow the enzyme to allow maximum time for the enzyme to oxidize the reducing sugars. Since the enzyme is dependent on availability of oxygen, the early addition is preferred in order to allow entrance of oxygen during milk processing and thus to allow a high as possible reduction of concentration levels of reducing sugars. The oxidoreductase according to the invention has a broad substrate specificity, allowing the oxidation of a broad range of reducing sugars. For dairy application in dairy products or dairy-containing food stuffs, the oxidoreductase according to the present invention is able to efficiently oxidize lactose, glucose and galactose

The enzyme can be contacted with more solid foodstuffs, for example cheese in several ways. The cheese may be contacted with the enzyme during the cheese making process by adding the enzyme at some stage in the making process (e.g. addition to the cheese milk) resulting in the incorporation of the enzyme in the cheese matrix. The enzyme will become active when oxygen is present; this may to some extent be in the cheese itself, but it will be most prominent during and/or after cheese processing such as slicing or grating which lead to significant increase in air-exposed cheese surface and oxygen exposure. Alternatively, the enzyme may be sprayed on the cheese containing foodstuff prior to heating, in this way preventing Maillard reactions on the surface which is the place where Maillard reactions are most prominently taking place. In this case the enzyme may be provided in a solution or a dispersion and sprayed on the foodstuff. The solution/dispersion may comprise the enzyme in amounts of 1-50 units OXI 01-OXI 06/ml. Alternatively, the enzyme may be added in a dry form, such as a powder.

The enzyme, either in dry or liquid form, may be added alone or in combination with other additives.

Surprisingly, it has been found that use of the oxidoreductase of the present invention can also decrease the growth of aerobic microorganisms in milk, thus contributing to the preservation of fresh milk. In such cases the oxidoreductase can be used as an anti-microbial agent in dairy applications, for example in milk, milk derived products or foodstuffs containing such products.

An additional advantage of the use of an oxidoreductase according to the present invention is that peroxides are formed. It is known that such peroxides can react with proteins, leading to protein crosslinking (see e.g. J. A. Gerrard, Trens in Food Science & technology (2002), 13, 391-399). However, the extent of cross-linking depends on the amount of hydrogen peroxide that is generated. Surprisingly, the oxidoreductase according to the present invention are capable of substantially crosslinking proteins in milk. The degree of crosslinking is also dependent on the pre-treatment of the substrate, the amount of oxygen available for oxidoreduction etcetera. The crosslinking of proteins has the advantage that the products comprising cross-linked proteins have altered textural properties, for example water holding capacity of gels formed from such cross-linked proteins.

### EXAMPLES

### Example 1

### Rheological Tests

The farinograph and extensograph are used by bakers worldwide, to evaluate the rheological and technological properties of dough.

The effect of the oxidoreductase on the rheological properties of the dough can be measured by standard methods according to the International Association of Cereal Chemistry (ICC) and the American Association of Cereal Chemistry (AACC) including the Rapid Visco Analyser, the farinograph method (AACC 54-2, ICC 115) and the extensograph (AACC 54-10, ICC 114).

In effect, the extensograph method measures the relative strength of dough. Strong dough exhibits a higher and, in some cases, a longer extensograph curve than does a weak dough. AACC method 54-10 defines the extensograph in the following manner: “the extensograph records a load-extension curve for a test piece of dough until it breaks. Characteristics of load-extension curves or extensograms are used to assess general quality of flour and its responses to improving agents”.

The farinograph method determines the water intake of a particular flour and the mixing tolerance of the resulting dough. Better baking flours, and dough, will exhibit higher farinograph values. If a particular flour shows relatively high water intake, and the mixing tolerance of the resulting dough is good, the farinograph curve shows retention of most if not all of the initial height over time. The machinability and baking quality of such a dough is likely to be excellent. AACC Method 54-12 defines the farinograph as follows: “the farinograph measures and records resistance of a dough to mixing. It is used to evaluate absorption of flours and to determining stability and other characteristics of doughs during mixing.”

### Example 2

### Measuring the Free Thiol Content of Dough

The effect of a redox enzyme on the formation of thiol group cross-linking can be studied by measuring the content of free thiol groups in a dough The method is described in Cereal Chemistry, 1983, 70, 22-26. This method is based on the principle that 5.5′-dithio-bis(2-nitrobenzoic acid) (DTNB) reacts with thiol groups in the dough to form a highly coloured anion of 2-nitro-5-mercapto-benzoic acid, which is measured spectrophotometrically at 412 nm.

### Example 3

### Mini-Batard Baking Test

Mini batards baking test was used for gluten strengthening. In order to detect the effect of enzymes on gluten strengthening the addition of chemical oxidizing agents was omitted. All tests are done in duplicate.

Recipe

| Ingredients | | |
|---|---|---|
| Flour Kolibi (Meneba) | 180 | g |
| Flour Ibis (Meneba) | 20 | g |
| Fresh yeast (Koningsgist) | 4.6 | g |
| Water 59% | 118 | g |
| Salt 2% | 4 | g |
| Fungal amylase Bakezyme P500 | 3 | ppm |
| Xylanase Bakezyme HSP6000 | 5 | ppm |

Process

| Process step | |
|---|---|
| Mixing | Pin mixer 6 min 15 sec |
| Scaling | 2 × 150 g |
| First proof | 25 min, room temperature |
| Moulding | Bertrand stick moulder adjustment 16 |
| Final proof | 90 min, 32° C., 85% RH |
| Baking | 20 min at 240/235° C., 0.2 l steam |

The height/width ratio is a measure for the stability of dough. When baking hearth bread, when no oxidizing agents are present, the dough is not stable and becomes flat and broad. The same characteristics are found in the final bread. The addition of enzymes that improve dough stability results in a higher height/width ratio.

During processing dough quality is evaluated by the baker. Height/width ratios are measured with a ruler. Results are evaluated statistically with ANOVA, Statgraphics plus 5.1.

### Example 3.1

Oxidoreductases having an amino acid sequence according to SEQ ID NO: 013, 014, 015, 016, 017 and 018 were tested in mini batards. All tests were done in duplicate. All enzymes were dosed as ultrafiltration concentrates and tested at 5 mg total protein per kg flour. The negative control was dough without addition of oxidoreductase. Ascorbic acid (68 mm) is taken along as a reference.

### Results:

| Addition | Height/width ratio |
|---|---|
| No oxidoreductase | 0.55 a* |
| SEQ ID NO: 013 | 0.65 c |
| SEQ ID NO: 014 | 0.59 b |
| SEQ ID NO: 015 | 0.59 b |
| SEQ ID NO: 016 | 0.59 b |
| SEQ ID NO: 017 | 0.60 b |
| SEQ ID NO: 018 | 0.61 b |
| Ascorbic acid | 0.66 c |

*Results with a different letter are statistically significant differences at the 90.0% confidence level. The method used to discriminate among the means is Fisher's least significant difference (LSD) procedure.
It is clearly seen that the loaves containing the oxidoreductases according to the invention show a better height/width ratio than the loaves not comprising these enzymes.

### Example 3.2

The dosage response of the enzyme comprising amino acid according to SEQ ID NO: 013 on the height/width ratio was tested in mini batards. Three dosages were tested: 1, 2 and 3 mg total protein/kg of flour. The negative control was dough without addition of oxidoreductase. Ascorbic acid (68 mm) is taken along as a reference. Tests are done in duplicate.

### Results:

| | Height/width ratio |
|---|---|
| Dosage SEQ ID NO: 013 | |
| (mg total protein/kg flour) | |
| 0 | 0.63 a* |
| 1 | 0.68 b |
| 2 | 0.70 bc |
| 3 | 0.72 c |
| Dosage ascorbic acid (mg/kg) | |
| 68 | 0.72 c |

*Results with a different letter are statistically significant differences at the 90.0% confidence level. The method used to discriminate among the means is Fisher's least significant difference (LSD) procedure.

It is found that the oxidoreductase according to the invention shows a nice dosage response in the height/width ratio of baked mini batards. Compared to the results of Example 3.1 the absolute values of the height/width ratios are higher in this test. This related to the fact that the test of example 3.1 is done with flour from the harvest of 2005 and example 3.2 with flour from the harvest of 2006.

1. An isolated polynucleotide hybridisable to a polynucleotide of any one of SEQ ID NO: 001-006 or SEQ ID NO: 007-012.

2. An isolated polynucleotide according to claim 1 hybridisable under high stringency conditions to a polynucleotide of any one of SEQ ID NO: 001-006 or SEQ ID NO: 007-012.

3. An isolated polynucleotide according to claim 1 obtainable from a filamentous fungus.

4. An isolated polynucleotide according to claim 3 obtainable from Aspergillus niger.

5. An isolated polynucleotide encoding an oxidoreductase comprising any one of amino acid sequences SEQ ID NO: 013-018 or functional equivalents of any of them.

6. An isolated polynucleotide encoding at least one functional domain of a oxidoreductase comprising any one of amino acid sequences SEQ ID NO: 013-018 or functional equivalents of any of them.

7. An isolated polynucleotide comprising any one of nucleotide sequences SEQ ID NO: 001-006 or SEQ ID NO: 007-012 or functional equivalents of any of them.

8. An isolated polynucleotide consisting of any one of SEQ ID NO: 001-006 or SEQ ID NO: 007-012.

9. A vector comprising a polynucleotide sequence according to claim 1.

10. A vector according to claim 9 wherein said polynucleotide sequence is operatively linked with regulatory sequences suitable for expression of said polynucleotide sequence in a suitable host cell.

11. A vector according to claim 10 wherein said suitable host cell is a filamentous fungus.

12. A method for manufacturing a polynucleotide according to claim 1 or a vector comprising said polynucleotide comprising the steps of culturing a host cell transformed with said polynucleotide or said vector and isolating said polynucleotide or said vector from said host cell.

13. An isolated oxidoreductase with an amino acid sequence SEQ ID NO: 013-018 or functional equivalents of any of them.

14. An isolated oxidoreductase according to claim 13 obtainable from Aspergillus niger.

15. An isolated oxidoreductase obtainable by expressing a polynucleotide according to claim 1 or a vector comprising said polynucleotide in an appropriate host cell, e.g. Aspergillus niger.

16. Recombinant oxidoreductase comprising a functional domain of any of the oxidoreductase according to claim 13.

17. A method for manufacturing a oxidoreductase according to claim 13 comprising the steps of transforming a suitable host cell with an isolated polynucleotide according to claim 1 or a vector comprising said polynucleotide, culturing said cell under conditions allowing expression of said polynucleotide and optionally purifying the encoded polypeptide from said cell or culture medium.

18. A recombinant host cell comprising a polynucleotide according to claim 1 or a vector comprising said polynucleotide.

19. A recombinant host cell expressing a polypeptide according to claim 13.

20. Purified antibodies reactive with an oxidoreductase according to claim 13.

21. A process for the production of dough comprising adding an oxidoreductase according to claim 13.

22. A process for the production of a baked product from a dough as prepared by the process of claim 21.

23. Use of an oxidoreductase according to claim 13 for the preparation of a dough and/or the baked product thereof.

24. Use of an enzyme capable of hydrolysing a hydroxy fatty acid for the preparation of a dough and/or the baked product thereof.

25. Use of an oxidoreductase according to claim 13 for the preparation of dairy products.

26. Use of an oxidoreductase according to claim 13 for the reduction of Maillard reactions in dairy products.

27. Use of an oxidoreductase according to claim 13 for the prevention of Maillard reactions in dairy products.

28. Use of an oxidoreductase according to claim 13 as an anti-microbial agent in dairy products.

29. Use of an oxidoreductase according to claim 28, characterized in that the anti-microbial agent is used for the lactoperoxidase-thiocyanate system in milk.

30. Use of an oxidoreductase according to claim 25, characterized in that the dairy products are milk or cheese.
